SEQUENCE LISTING (66 sequences)

SEQ ID NO: 1 - protein, 214 aa, unidentified
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 2 - protein, 214 aa, unidentified
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSGKTTMPLW

SEQ ID NO: 3 - protein, 214 aa, unidentified
MKLLILTCLVAVALARPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 4 - protein, 214 aa, unidentified
MKLLILTCLVAVALARPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 5 - protein, 177 aa, unidentified
MKFLILTCLVAVALARPKLPLRHPELTQNELDSREPTPRELREEYFSELSKRELLREKQNEGIKVQVMEDPEQRQSSSTSSSEEVVPNNTEEQLRRLSQHNQLQVGTIHDQQQLRRVNENNLLQLPFQQFYQLDAYPYAAWYFPAQIMQYIAYPPSLDITKPIASENIENADVVPQW

SEQ ID NO: 6 - protein, 206 aa, unidentified
MKLLIFICLAAVALARPKPPLRHQEHLQNEPDSREELFKERKFLRFPEVPLLSQFRQEIINELNRNHGMEGHEQRGSSSSSSEEVVGNSAEQKHVQKEEDVPSQSYLGHLQGLNKYKLRQLEAIHDQELHRTNEDKHTQQGEPMKGVNQEQAYFYFEPLHQFYQLDAYPYATWYYPPQYIAHPLFTNIPQPTAPEKGGKTEIMPQW

SEQ ID NO: 7 - protein, 185 aa, unidentified
MRLLILTCLVAVALARPKLPLRYPERLQNPSESSEPIPLESRQEYMNGMNRQRNILREKQTDEIKDTRNESTQNCVMAEPEKMESSISSSSEEISLSKCAEQFCRLNEYNQLQLQAVHAQEQIRRMNENSHVQVPFQQLNQLAAYPCAVWYYPQIMQYVPFPPFSDISNPTAHENYEKNNVMLQW

SEQ ID NO: 8 - protein, 230 aa, unidentified
MKLLILTCLVAVALARPKYPLRYPEVFQNEPDSIEEVLNKRKILELAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQEQAYFHLEPFPQFFQLGASPYVAWYYPPQVMQYIAHPSSYDTPEGIASEDGGKTDVMPQWW

SEQ ID NO: 9 - protein, 212 aa, unidentified
MKLLILTCLVAVALARPKLPHRHPEIIQNEQDSREKVLKERKFPSFALEYINELNRQRELLKEKQKDEHKEYLIEDPEQQESSSTSSSEEVVPINTEQKRIPREDMLYQHTLEALRRLSKYNQLQLQAIYAQEQLLRMKENSQRKPMRVVNQEQAYFYLEPFQPSYQLDVYPYAAWFHPAQIMQHVAYSPFHDTAKLIASENSEKTDIIPEW

SEQ ID NO: 10 - protein, 185 aa, unidentified
MRLLILTCLVAVALARPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 11 - protein, 199 aa, unidentified
MKLLIFSCLMALALARPDVLHLSIDRHIKHREVENRSNEDLIPLNEVSSSEESLHQLNRDRRSPEKYELNKYREDLKTSSSEESVAPSTEESVRRQVEYNFNEQEDASASRERKIEDVSEQYRQYLRRRPEERALNLRYLEPLYYATEPDFYYTYVPISMPRFFPYPAEAPVFSTRKAPVPSINRATEAVYTYSEEKKN

SEQ ID NO: 12 - protein, 181 aa, unidentified
MKVLILACLVAVAVAMPESPSSSSSSEEAPRLLTKKRILRNQEYYLPHLEESRSSSSSEESTRPTLKRLLLKEKPILHILKAPESSSSEESDSAAEKRLLREREFYQQQLDEYYRQFEPDFYPRAYPKKEVMPYPLEYFIPQAAVYSIPQLVYRVPQEVTFPSPLRFRYAFPQPTLPVERK

SEQ ID NO: 13 - protein, 183 aa, unidentified
MKVLILACLVAFVVAMPESPSSSSSSEEASKILTKKRVQRDQEYYLPHQEESVSSSSSEESTDRLKRRLLKDKPIFRLLKATESSSSEESDSAIEKRILRERQYYQQKLDELKEYFRQFEPYFYPVAYQKKEVMPYQLEYFVPQPEVYSIPQPVYRVPQEVTFPSLLHFRYAFPQSTLPIERK

SEQ ID NO: 14 - protein, 215 aa, unidentified
MKLLILTCLVATALARHKFHLGHLKLTQEQPESSEQEILKERKLLRFVQTVPLELREEYVNELNRQRELLREKENEEIKGTRNEVTEEHVLADRETEASISSSSEEIVPSSTKQKYVPREDLAYQPYVQQQLLRMKERYQIQEREPMRVVNQELAQLYLQPFEQPYQLDAYLPAPWYYTPEVMQYVLSPLFYDLVTPSAFESAEKTDVIPEWLKN

SEQ ID NO: 15 - protein, 313 aa, unidentified
MKLLILTCLVAAAFAMPRLHSRNAVSSQTQQQHSSSEEIFKQPKYLNLNQEFVNNMNRQRALLTEQNDEIKVTMDAASEEQAMASAQEDSSISSSSEESEEAIPNITEQKNIANEDMLNQCTLEQLQRQFKYNQLLQKASLAKQASLFQQPSLVQQASLFQQPSLLQQASLFQQPSMAQQASLLQQLLLAQQPSLALQVSPAQQSSLVQQAFLAQQASLAQKHHPRLSQSYYPHMEQPYRMNAYSQVQMRHPMSVVDQALAQFSVQPFPQIFQYDAFPLWAYFPQDMQYLTPKAVLNTFKPIVSKDTEKTNVW

SEQ ID NO: 16 - protein, 198 aa, unidentified
MKLLILTCLVASAVAMPKFPFRHTELFQTQRGGSSSSSSSEERLKEENIFKFDQQKELQRKQSEKIKEIISESTEQREASSISSSEEVVPKNTEQKHIPQEDALYQQALEQLSRLIKYHQLQMEVVHAQEQFHRINEHNQAQVKEPMRVFNQLDAYPFAAWYYGPEVQYMSFLPFSSIPQPIFPEDAQNTEVMPEWVM

SEQ ID NO: 17 - protein, 222 aa, unidentified
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 18 - protein, 222 aa, unidentified
MKFFIFTCLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVITKKTKLTEEDKNRLNFLKKISQHYQKFTWPQYLKTVYQYQKAMKPWTQPKTNVIPYVRYL

SEQ ID NO: 19 - protein, 223 aa, unidentified
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 20 - protein, 223 aa, unidentified
MKFFIFTCLLAVALAKHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 21 - protein, 235 aa, unidentified
MKFFIFTCLLAVAFAKHEMEHVSSSEESINISQEKYKQEKNVINHPSKEDICATSCEEAVRNIKEVGYASSSSSEESVDIPAENVKVTVEDKHYLKQLEKISQFYQKFPQYLQALYQAQIVMNPWDQTKTSAYPFIPTVIQSGEELSTSEEPVSSSQEENTKTVDMESMEEFTKKTELTEEEKNRIKFLNKIKQYYQKFTWPQYIKTVHQKQKAMKPWNHIKTNSYQIIPNLRYF

SEQ ID NO: 22 - protein, 193 aa, unidentified
MKFFIFTCLLAVVLAKHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFTKKTELTEEEKDHQKFLNKIYQYYQTFLWPEYLKTVYQYQKTMTPWNHIKRYF

SEQ ID NO: 23 - protein, 236 aa, unidentified
MKFFIFTCLLAVALAKHNMEHRSSSEDSVNISQEKFKQEKYVVIPTSKESICSTSCEEATRNINEMESAKFPTEVYSSSSSSEESAKFPTEREEKEVEEKHHLKQLNKINQFYEKLNFLQYLQALRQPRIVLTPWDQTKTGASPFIPIVNTEQLFTSEEIPKKTVDMESTEVVTEKTELTEEEKNYLKLLNKINQYYEKFTLPQYFKIVHQHQTTMDPQSHSKTNSYQIIPVLRYF

SEQ ID NO: 24 - protein, 223 aa, unidentified
MKLFIFTCLLAVALAKHKSEQQSSSEESVSISQEKFKDKNMDTISSEETICASLCKEATKNTPKMAFFSRSSSEEFADIHRENKKDQLYQKWMVPQYNPDFYQRPVVMSPWNQIYTRPYPIVLPTLGKEQISTIEDILKKTTAVESSSSSSTEKSTDVFIKKTKMDEVQKLIQSLLNIIHEYSQKAFWSQTLEDVDQYLKFVMPWNHYNTNADQVDASQERQA

SEQ ID NO: 25 - protein, 182 aa, unidentified
MKFFIFTCLLAVALAKPKIEQSSSEETIAVSQEVSPNLENICSTACEEPIKNINEVEYVEVPTEIKDQEFYQKVNLLQYLQALYQYPTVMDPWTRAETKAIPFIRTMQYKQEKDATKHTSQKTELTEEEKAFLKYLDEMKQYYQKFVFPQYLKNAHHFQKTMNPWNHVKTIIYQSVPTLRYL

SEQ ID NO: 26 - protein, 184 aa, unidentified
MKFFIFACLVVVALAKHEIKDKSSSEESSASIYPGKSKLDNSVFFQTTKDSASSSSSEESSEEVSEKIVQSEEQKVNLNQQKKFKQFSQESSFSQCCTPLHQQQQSSVNQWPQPNAIHNTPTQESISTSVEEILKKIIDMIKYIQYQQVTIPQLPQALHPQIPVSYWYPSKDYTFPNAHYTRFY

SEQ ID NO: 27 - protein, 179 aa, unidentified
MKFFIFTCLVAAALAKHAVKDKPSSEESASVYLGKYKQGNSVFFQTPQDSASSSSSEESSEEISEKIEQSEEQKVNLNQQKKSKQFSQDSSFPQICTPYQQQSSVNQRPQPNAIYDVPSQESTSTSVEEILKKIIDIVKYFQYQQLTNPHFPQAVHPQIRVSSWAPSKDYTFPTARYMA

SEQ ID NO: 28 - protein, 180 aa, unidentified
MRFFVFTCLLAVALAKNGIEQRSASEEIVSFYQEKYKQDSNAAIYPTNQETPSVSSSEESVEVQTEKDEQIEEENVYLKQLKRIKQIFQKFYIPQYPEVYQQQIVMNPWKHVKTTTYPVPIPETTRIPLEEIVKKIVEMIKFNQLHQFVIPQYVQALQQRIAMNPWHHVTPFRSFPVLNF

SEQ ID NO: 29 - protein, 143 aa, unidentified
MKFIILTCLLAVALAKQRMEQYISSEESMDNSQENFKQNMDVAFFPSQETVENIYIPQMESVEAPMKVSDIISQQQYNQKMMDMSVSAREKTVMTEESKNIQDYMNKMKRYSKITWPQFVKLLHQYQKTMTPWSYYPSTPSQV

SEQ ID NO: 30 - protein, 169 aa, unidentified
MKFIILTCLLAVALAKQESKDNSQEDFKQTVDVVIFPGQETVKNIPIPQMESVEAPIKNKCYQSIQTFKPPQALKGLYQYHMAKNPWGYTVNRAFPSTRTLQYNQKTMDLSMRAREKIVMSEIKKNIQDYVTKMKQYSKITWPRFVKSLQQYQKTMNPWSCYPYTLLQV

SEQ ID NO: 31 - protein, 177 aa, unidentified
MKVFILACLVAAAVAVPVSTEFDKLLVKEKLLKHRDLVKDLPTIFSSEWEQFLRHPEVYVPLEKYQQRLRLFKPTYLVPVNKFVERHPFRNILFPEELPEAYQPIEKEDSSSSSEETVQVPVEKHLLRLRKLHVPQKLRPLRFYPNHQVPFXRHPLPYAGTQVHQPVEVPFPLPXQY

SEQ ID NO: 32 - protein, 189 aa, unidentified
MKVFIFACLVAVAMAVPKQQSSSSSSEETDKQLVMENLLKHRALVKDIPTTFSSEENINYEKQWEQLLRQPMVYEPFEIYQQGLRPFKPTHLRRPLKYIFFSEEPPKVYQPIQNEDSSSSSEEPVEVPAEQNHVLRLKKLQVLQNLQPLRRLPNYQVPLQRHPLPFVRLPNVFQAPHPVELPFPLPQVV

SEQ ID NO: 33 - protein, 224 aa, unidentified
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 34 - protein, 224 aa, unidentified
MKVLILACLVALALARELEELNVPGEIVESLSSSEESITHINKKIEKFQSEEQQQMEDELQDKIHPFAQTQSLVYPFPGPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 35 - protein, 222 aa, unidentified
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGPFPILV

SEQ ID NO: 36 - protein, 222 aa, unidentified
MKVLILACLVALALAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLPPTVMFPPQSVLSLSQPKVLPVPQKAVPQRDMPIQAFLLYQEPVLGPVRGPFPILV

SEQ ID NO: 37 - protein, 232 aa, unidentified
MKLLILACFVALALARAKEELNASGETVESLSSSEESITHISKEKIEKLKREEQQQTENERQNKIHQFPQPQPLAHPYTEPIPYPILPQNILPLAQVPVVVPLLHPEVMKDSKAKETIVPKRKGMPFPKSPAEPFVEGQSLTLTDFEVLSLPLLQSLMHQIPQPVPQTPMFAPQPLLSLPQAKVLPVPQQVVPFPQRDMPFQALLLYQDPLLGPLQGFYPVPQPVAPVYNPV

SEQ ID NO: 38 - protein, 232 aa, unidentified
MKVLILACRVALALAREKEEFKTAGEALESISSSEESITHINKQKIEKFKIEEQQQTEDEQQDKIYTFPQPQSLVYSHTEPIPYPILPQNFLPPLQPAVMVPFLQPKVMDVPKTKETIIPKRKEMPLLQSPVVPFTESQSLTLTDLENLHLPLPLLQSLMYQIPQPVPQTPMIPPQSLLSLSQFKVLPVPQQMVPYPQRAMPVQAVLPFQEPVPDPVRGLHPVPQPLVPVIA

SEQ ID NO: 39 - protein, 241 aa, unidentified
MKILILACLVALALAREKEELNVSSETVESLSSNEPDSSSEESITHINKEKLQKFKHEGQQQREVERQDKISRFVQPQPVVYPYAEPVPYAVVPQSILPLAQPPILPFLQPEIMEVSQAKETILPKRKVMPFLKSPIVPFSERQILNPTNGENLRLPVHLIQPFMHQVPQSLLQTLMLPSQPVLSPPQSKVAPFPQPVVPYPQRDTPVQAFLLYQDPRLGPTGELDPATQPIVAVHNPVIV

SEQ ID NO: 40 - protein, 250 aa, unidentified
MKVFILACLVALALAREKEELTLSNETVESLSSSEESITHINKQKLENFKHEEQQQREDERQNKIHPLFQQQPLVSPYADPIHYAILPQNILPLAQPAVVVPFLQPEIMEVPKVKENIFPRHKVMPFLKSPVTPFLDSQILNVADLENVHFPLPLSLPLLQPLMHQIPQPLPLLQPLMHQIPQPLPQTPMLTPQSVLSIPQPKVLPFPQQVVPYLQRDMPLQAFLPYQESTHQAQPVTQPLAPLVNSALV

SEQ ID NO: 41 - protein, 226 aa, unidentified
MKVLILACLVALALARETIESLSSSEESITEYKQKVEKVKHEDQQQGEDEHQDKIYPSFQPQPLIYPFVEPIPYGFLPQNILPLAQPAVVLPVPQPEIMEVPKAKDTVYTKGRVMPVLKSPTIPFFDPQIPKLTDLENLHLPLPLLQPLMQQVPQPIPQTLALPPQPLWSVPQPKVLPIPQQVVPYPQRAVPVQALLLNQELLLNPTHQIYPVTQPLAPVHNPISV

SEQ ID NO: 42 - protein, 228 aa, unidentified
MKVLILACLVALALAREKEQLSVPTEAVGSVSSSEEITHINKQKLETIKHVEQLLREEKLQDKILPFIQSLFPFAERIPYPTLPQNILNLAQLDMLLPLLQPEIMEDPKAKETIIPKHKLMPFLKSPKTVPFVDSQILNLREMKNQHLLLPQLLPFMHQVFQPFPQTPIPYPQALLSLPQSKFMPIVPQVVPYPQRDMPIQALQLFQELLFPTHQGYPVVQPIAPVNV

SEQ ID NO: 43 - protein, 231 aa, unidentified
MKVFILACLVALALAREKDAFTVSSETGSISSEESVEHINEKLQKVKLMGQVQSEDVLQNKFHSGIQSEPQAIPYAQTISCSPIPQNIQPIAQPPVVPTVGPIISPELESFLKAKATVLPKHKQMPFLNSETVLRLFNSQIPSLDLANLHLPQSPAQLQAQIVQAFPQTPAVVSSQPQLSHPQSKSQYLVQQLAPLFQQGMPVQDLLQYLDLLLNPTLQFLATQQLHSTSV

SEQ ID NO: 44 - protein, 231 aa, unidentified
MKVFILACLVALALARETTFTVSSETDSISSEESVEHINEQKLQKVNLMGQLQAEDVLQAKVHSSIQSQPQAFPYAQAQTISCNPVPQNIQPIAQPPVVPSLGPVISPELESFLKAKATILPKHKQMPLLNSETVLRLINSQIPSLASLANLHLPQSLVQLLAQVVQAFPQTHLVSSQTQLSLPQSKVLYFLQQVAPFLPQDMSVQDLLQYLELLNPTVQFPATPQHSVSV

SEQ ID NO: 45 - protein, 239 aa, unidentified
MKVFILACLVAFALGREKEEIIVSTETVENLSSSEIRQFYSEESVTQVNKQKPEGVKHEEQQREDEHQNKIQPLFQPQPLVYPFAEPIPYTVFPPNAIPLAQPIVVLPFPQPEVKQLPEAKEITFPRQKLMSFLKSPVMPFFDPQIPNLGTDLENLHLPLPLLQPLRHQLHQPLAQTPVLPLPLSLPKVLPVPQQVIPYPQRGRPIQNLQLYEEPLLDPTRKIYPVAQPLAPVYNPVAV

SEQ ID NO: 46 - protein, 285 aa, unidentified
MKLLILTCLVVLAVARPMVEKISESEEHVTDVPENEHRLEINRYLRPEYEMMNLYYQPFYWSEEMRNLKMTSLPKDRRMAVLKSVVSDDMLPPLQHKSLSLPKPKVLPLSHRQILPPHTLRMVPLSHKLFTIPKREMLPISERERLPAHERENLLAHEREILLAPQREMSLIPEREILLAAERVVLPEQEREIRPDNEREVLAVHKREILPASEKEKVLPLFQERVLPLHRREIVPPYQRDTIARREILPVDQRELMPEVVAVDLYPFFQPVANFYYPAELNEKN

SEQ ID NO: 47 - protein, 189 aa, unidentified
MKVFILSCLLAVAMAMPKLQSSSSSSEETDQLLVKEKLVKRRELMDLPTTLSSEEHVMEEKEFYQPRLKYPYPFFPPIKTYVNPHIYQKPAVLPVTHPETLTYLQPQQNPEDMPLPKKEVLPYLKAVVVPYPQVQVMPYPETEVMPYFPPMTMSLVQPDIVPPSFYREAVIRPVAYNLPPTVQKIPETN

SEQ ID NO: 48 - protein, 183 aa, unidentified
MKVFILACLVAVAMALPKQHSSSSSSEESDRLLVKDIPTAFSSEEHSVDPKELYEPRQSYSYPWQSVRPINTYTYPRAYQIPAVLPMTHPQTLTYLQPQFKPEDMSISQKQIPPYVQAVVMPYPQVEAIPFPGAEFMPYAQPITTPLLQPEVFSAPFYREAVFKPVIYGLPQSQPVQKIPETD

SEQ ID NO: 49 - protein, 165 aa, unidentified
MKVFIFACLVAVAMAVPKQQSSSSSSEETDKQLVMENLLKHRALVENINYEKQWEQLLRQPMVYEPFERRPLKYIFFSEEPPKVYQPIQNEDSSSSSEEPVEVPAEQNHVLRLKKLQVLQNLQPLRRLPNYQVPLQRHPLPFVRLPNVFQAPHPVELPFPLPQVV

SEQ ID NO: 50 - protein, 190 aa, unidentified
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 51 - protein, 190 aa, unidentified
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSEVIESVPETNTAQVTSTVV

SEQ ID NO: 52 - protein, 192 aa, unidentified
MMKSFFLVVTILALTLPFLGAQEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 53 - protein, 192 aa, unidentified
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 54 - protein, 182 aa, unidentified
MKSFFLVVTILALTLPFLGAEVQNQEQPTCFEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 55 - protein, 188 aa, unidentified
MMKSSFLIVPILALTLPFLGAEEQNQEKLTRCESDKRLFNEEKVKYIPIYYMLNRFPSYGFFYQHRSAVSPNRQFIPYPYYARPVVAGPHAQKPQWQDQPNVYPPTVARRPRPHASFIAIPPKKNQDKTAIPAINSIATVEPTIVPATEPIVNAEPIVNAVVTPEASSEFLITSAPETTTVQVTSPVV

SEQ ID NO: 56 - protein, 180 aa, unidentified
MMKHFLLVVNILAVTLPFLAADIQNQEQTTCRENEERLFHQVTAPYIPVHYVMNRYPQYEPSYYLRRQAVPTLNPFMLNPYYVKPIVFKPNVQVPHWQILPNIHQPKVGRHSHPFFMAILPNKMQDKAVTPTTNTIAAVEPTPIPTTEPVVSTEVIAEASPELIISPETTTEATAASAAA

SEQ ID NO: 57 - protein, 234 aa, unidentified
MMKSFLLVVNIVALTLPFLAAEVQNQEQPACCGNDERLFEQKKVLYLLSYPVLNNYLRTAPSYYQNRASVPINNPYLCHLYYVPSFVLWAQGQIPKGPVSTDIHQSTMQYHQAKHPSFMAILSKKILGKATILSTDAIAAPEQTPVSAAQPTVSAGDTPEVSSQFIDTPDTSVLAEEARESPEDTPEISEFINAPDTAVPSEEPRESAEDTPEISSEFIFSPETSTGPAIASMA

SEQ ID NO: 58 - protein, 178 aa, unidentified
MMRNFIVVMNILALTLPFLAAEVQNPDSNCREKNEVVYDVQRVLYTPVSSVLNRNHYEPIYYHYRTSVPVSPYAYFPVGLKLLLLRSPAQILKWQPMPNFPQPVGVPHPIPNPSFLAIPTNEKHDNTAIPASNTIAPIVSTPVSTTESVVNTVANTEASTVPISTPETATVPVTSPAA

SEQ ID NO: 59 - protein, 181 aa, unidentified
MMRNFIVVVNILALTLPFLAAEIQNPDSNCRGEKNDIVYDEQRVLYTPVRSVLNFNQYEPNYYHYRPSLPATASPYMYYPLVVRLLLLRSPAPISKWQSMPNFPQSAGVPYAIPNPSFLAMPTNENQDNTAIPTIDPITPIVSTPVPTMESIVNTVANPEASTVSINTPETTTVPVSSTAA

SEQ ID NO: 60 - protein, 182 aa, unidentified
MKSFLLVVNALALTLPFLAVEVQNQKQPACHENDERPFYQKTAPYVPMYYVPNSYPYYGTNLYQRRPAIAINNPYVPRTYYANPAVVRPHAQIPQRQYLPNSHPPTVVRRPNLHPSFIAIPPKKIQDKIIIPTINTIATVEPTPAPATEPTVDSVVTPEAFSESIITSTPETTTVAVTPPTA

SEQ ID NO: 61 - protein, 185 aa, unidentified
MKSFFLVVNILALTLPFLGAEVQNQEQPTCHKNDERFFDLKTVKYIPIYYVLNSSPRYEPIYYQHRLALLINNQHMPYQYYARPAAVRPHVQIPQWQVLPNIYPSTVVRHPCPHPSFIAIPPKKLQEITVIPKINTIATVEPTPIPTPEPTVNNAVIPDASSEFIIASTPETTTVPVTSPVVQKL

SEQ ID NO: 62 - protein, 185 aa, unidentified
MMKRFFLVVNIVALALPFLQGAEVQNQEQPTCRENDERLFNQKTVKYIPIHYVLNSFSHYEPNYYPHRPAEPINHQYVPYPFYAKPAVAVRTHAQIPQWQVLPNAYPPTMMHRPQLHPSFIAIPPKKIQDKTSIPTINTIATAEATPIPLTEPKVNTAVTSDASSEFTITSTPETTTVPVTSPVV

SEQ ID NO: 63 - protein, 181 aa, unidentified
MMKGFLLVVNILLLPLPFLAAEVQNQEESRSCCLEKDERWFCQKAVKYIPNDYVLKSYYRYEPNYNQFRAAVPINNPYLIYLYPAKQVAVRPHTQIPQWQVPSNIYPSPSVPHTYLKPPFIVIPPKKTQDKPIIPPTGTVASIEATVEPKVNTVVNAEASSEFIATNTPEATTVPVISPQI

SEQ ID NO: 64 - protein, 167 aa, unidentified
MKTLLLVGAILAMTVGFSVAEEQKWKRLDSSESEERWWRLRLKPSLLFRVQDKPERNIPRPSYPYPLLNVPHPNAINPEHQRPYVLPRFNFQIPNILPFLMFPELPPPFFPIVHPIYYDPQTPTTPRNPPVTSQTPQPPVDSSANTPEPPTTAPLTATPEAQTPLQP

SEQ ID NO: 65 - protein, 165 aa, unidentified
MKTLLLVGGILVMTVCFSAAEDEEWKKVDYSESEERWLRLKRQPSFPFSFQGKPERNIPRPYYPRPFLNIPRPYTINPEHQFAYVFPNLKFQIPSVFPFPLEFLPPFYPFVHPIYYGPQTSTPPRNPTVTSQTPQPPVHSSANTPESATAAPVTATPMAQTPLQP

SEQ ID NO: 66 - protein, 178 aa, unidentified
MKVLFLTVHILAVMVCFSTADLDWEKWPCDKQNERQSELRQQPLRRSPVQYVYTPYTHQSYVPVIYPPRAYVRHPYFSRVAWQKPYPSYMPLLPSIYPWSVVSRNLHPAFAFNPPHYAQLPVPSSPTNSPTTTIQTTNIPITNPTSTIVTPAVSSKSAATEDSAAAAMLTSPTAAQMA

(19) United States
(12) Patent Application Publication
(10) Pub. No.: US 2025/0344719 A1
(43) Pub. Date: Nov. 13, 2025

(54) COMPOSITIONS

(71) Applicant: ALL G CO HOLDINGS PTY LIMITED, Waterloo, New South Wales (AU)

(72) Inventors: Jared Raynes, Waterloo (AU); Isabella Loughland, Waterloo (AU); Carl Holt, Dalrymple (GB)

(73) Assignee: ALL G CO HOLDINGS PTY LIMITED, Waterloo, New South Wales (AU)

(21) Appl. No.: 18/869,176

(22) PCT Filed: May 30, 2023
(86) PCT No.: PCT/AU2023/050454

§ 371 Date: Nov. 25, 2024

(30) Foreign Application Priority Data
May 30, 2022 (AU) 2022901459

(51) Int. Cl. A23J 1/00; A23C 9/13; A23C 11/06; A23C 20/00; A23J 1/18; A23J 1/20; C12P 21/00

(52) U.S. Cl. CPC A23J 1/008; A23C 9/1315; A23C 11/065; A23C 20/00; A23J 1/18; A23J 1/202; C12P 21/00

(57) ABSTRACT

This invention provides a casein micelle composition containing amorphous calcium phosphate (CaP), wherein the casein micelle comprises at least one calcium sensitive casein, and wherein the casein micelle does not contain κ-casein. The present invention also provides methods for the production of casein micelle compositions and. The invention also provides food products comprising the casein micelles compositions.

### PRIORITY DETAILS

This application claims priority from Australian Patent Application No. 2022901459 filed on 30 May 2022, the contents of which are to be taken as incorporated herein by this reference.

### TECHNICAL FIELD

The present invention relates to casein micelle compositions and methods for their production. The invention also relates to food products comprising the casein micelle compositions.

### BACKGROUND OF INVENTION

With global warming continuing and the world's population predicted to reach 10 billion by 2050, there is an urgent global need for the sustainable production of high-quality protein whilst at the same time reducing greenhouse gas emissions (GHGE) from animal production.

Dairy proteins are one of most nutritious sources of protein because of their amino acid profile, inherent ability to transport high concentrations of bioavailable calcium and phosphate through the protein assembly known as the casein micelle and their digestibility in the stomach. A current major drawback of traditional dairy farming is the high environmental cost of producing milk in terms of land and water usage and the amount of GHGE produced, in particular methane.

Plant-based dairy alternatives whilst having lower GHGE and lower water usage do not match the nutrition, functionality and taste of animal-based dairy due to them not containing casein proteins, a class of proteins that only occur in mammals. The protein component of mammalian milk is made up of caseins and whey proteins. Caseins are intrinsically disordered proteins (IDPs), sometimes glycosylated and often phosphorylated, whereas whey proteins are more globular with a well-defined folded conformation. Caseins are further divided into calcium sensitive caseins and caseins insensitive to precipitation by calcium ions. The calcium-sensitive caseins in the cow are the α_{S1}-, α_{S2}- and β-caseins, whereas κ-casein is insensitive to precipitation by calcium ions.

Cellular agriculture is an alternative means of traditional agriculture that includes culturing of muscle and fat cells to replace slaughtered animal meat and engineering microorganisms to express a range of food molecules, in particular animal proteins such as those found in dairy and eggs. Using cellular agriculture to produce casein proteins through the genetic engineering of microorganisms is a potential approach which mitigates the environmental drawbacks of animal agriculture. Due, however, to the caseins being intrinsically disordered proteins, as well as their high levels of post translational modifications including phosphorylation and glycosylation, they are extremely difficult to produce economically and to also reassemble into native-like casein micelles, which is needed to produce stable dairy products such as milk.

Natural casein micelles are formed in the mammary gland. When combined with nanoclusters of amorphous calcium phosphate (CaP), they form stable, polydisperse supramolecules. The white appearance of milk is due to the intense scattering of light by casein micelles and fat globules.

Milk and natural casein micelles exhibit great stability compared to other biofluids and globular proteins, respectively. Milk can be stored in the mammary gland for days, weeks, or even months in some species, without aggregating or forming amyloid fibrils. Milks can withstand pasteurisation and more extreme heat treatments, they can be dried and afterwards reconstituted in water. Moreover, although many milks contain high concentrations of calcium and phosphate, greatly exceeding the solubility of calcium phosphates at milk pH, none of these treatments will normally produce a precipitate of calcium phosphate. These natural properties are exploited in the manufacture and storage of a range of liquid dairy products.

Although stable at milk pH, casein micelles readily aggregate to form a gel at acid pH. Likewise, the stability of the casein micelles may be reduced or eliminated by limited proteolysis catalysed by an aspartate proteinase such as chymosin, or similar proteinase. This ability is readily exploited in the manufacture of dairy gel products such as cheese and yogurt.

There are large natural variations in the composition of casein micelles among different species. For example, in cow's milk there are secreted protein polymorphs from four expressed casein genes, κ-, β-, α_{S1}- and α_{S2}-in approximate ratios of ˜1:4:4:1, respectively. In contrast, elephant milk contains expressed and secreted proteins from only two casein genes, κ- and β-casein in a ratio of ˜1:8.5, respectively. In the milk of some other species such as the rabbit, there are proteins from 5 casein genes. All milks that have been sufficiently well-characterised contain a form of κ-casein but all other caseins may be present or absent in different mammalian species.

While the naturally occurring casein micelles vary widely, there are no known examples of casein micelles made from a single casein. Thus, there currently exists a conserved minimum number of two casein proteins needed to form a casein micelle. All naturally occurring casein micelles contain a type of κ-casein and one or more types of calcium-sensitive casein. A type of κ-casein is present in the milk of all mammalian species. Moreover, κ-casein has generally been considered important for the stability of milk, by reason of its lack of sensitivity to precipitation by divalent cations such as Ca²⁺. It is widely accepted that the stability of casein micelles is due to a mechanism of stabilising colloidal particles known as steric stabilisation.

The fraction of total casein that is κ-casein varies widely. Within the Equidae, low proportions of κ-casein are found, for example 1.8% of horse milk casein and 2.8% in donkey. The proportion can vary widely among individuals and through lactation. For example, in a recent survey of lactational variation of casein composition in individual women, the proportion of κ-casein ranged from 11.5 to nearly 76% of total casein.

While there are limited examples in the prior art which claim to have made single casein micelles, all examples examined thus far use commercially sourced casein proteins. Commercially available casein proteins are invariably contaminated with other casein proteins, and particularly κ-casein, which is previously described to help with micelle stability. Hence, there are no examples in the prior art of truly single casein micelles.

Artificial casein micelles are not inevitable. When mixing caseins and salts there is a variety of possible outcomes, as shown in FIG. 1. The outcome depends on several factors, among which are the final composition of the formulation, the order in which ingredients are added and the rate at which additions are made. Moreover, the stability of the formulation can depend on how long after mixing the judgement is made.

Hydrophobic substances, such as the various minerals of low solubility identified in FIG. 1, can be stabilised in a colloidal state by adsorption of a more hydrophilic coat. The phenomenon leads to coat-core structures that may be kinetically stable and endure for long periods of time under favourable circumstances. However, with CaP or CaCO₃, the first-formed amorphous phase is also stabilised by casein against maturing into a less soluble and more crystalline form of CaP so there are two mechanisms of destabilisation of the colloid which are both affected by the concentration of casein and the affinity of the casein for binding to the amorphous and more crystalline phases. Destabilisation can also result from the loss of solubility of the casein, for example by salting out or charge reduction. To establish what has resulted from a particular formulation and mixing procedure, various methods of characterisation can be used to establish the composition, size and substructure of the product and its stability.

Verification that a casein micelle structure has been formed requires evidence from chemical analysis of the colloid and continuous phase that both calcium (Ca) and phosphate (Pᵢ) are present in the colloid. Appropriate chemical analysis includes Nanoparticle Tracking Analysis (NTA) size data, turbidity before and after centrifugation, Transmission Electron Microscopy (TEM) and mineral analysis. Evidence that the colloid contains CaP nanoclusters can also be shown using, for example cryo-electron microscopy or small-angle scattering experiments with X-rays or neutrons.

There is therefore an ongoing need for improved artificial casein micelle compositions and methods for their preparation, which at least partially address one or more of the above-mentioned short-comings or provides a useful alternative.

Particularly, there is an ongoing need for improved artificial casein micelle compositions which resemble the natural casein micelles of milk in their structure, composition and physico-chemical properties. These may include, for example, some of the following properties: (i) the white appearance and high calcium concentration of solutions; (ii) their stability at neutral pH to heat treatments or drying; (iii) avoidance of amyloid fibril formation; (iv) gelation on acidification; or (v) gelation after limited proteolysis.

A reference herein to a patent document or other matter which is given as prior art is not to be taken as an admission that the document or matter was known or that the information it contains was part of the common general knowledge as at the priority date of any of the claims.

### SUMMARY OF INVENTION

In a first aspect, the invention provides a composition comprising casein micelle particles containing amorphous calcium phosphate (CaP), wherein the casein micelle particles comprise at least one calcium sensitive casein, and wherein the casein micelle does not contain κ-casein.

In different aspects of the invention, the casein micelle particles comprise: (i) only αs1-casein; (ii) only αs2-casein; (iii) only β-casein; (iv) a mixture of αs1- and β-casein; (v) a mixture of αs2- and β-casein; (vi) a mixture of αs1- and αs2-casein; or (vii) a mixture of αs1-, αs2- and β-casein.

Advantageously, the casein micelle compositions of the present invention contain no κ-casein and yet present a similar stability and appearance to naturally occurring casein micelles. This allows casein micelle compositions to be produced which contain only calcium sensitive caseins and, in some aspects, only one casein protein.

Furthermore, the casein micelles of the present invention can be produced more economically than current solutions. In a further aspect, the present invention relates to methods for producing the casein micelle compositions disclosed herein.

The food products of the present invention can advantageously provide improved plant-based dairy alternatives. These food products provide improved nutrition, functionality and/or taste while avoiding some or all of the drawbacks associated with both animal-based dairy products and with current plant-based dairy alternatives. In a still further aspect, the present invention relates to food products comprising the casein micelle compositions disclosed herein.

Further aspects of the invention appear below in the detailed description of the invention.

### BRIEF DESCRIPTION OF DRAWINGS

Embodiments of the invention will herein be illustrated by way of example only with reference to the accompanying drawings in which:

FIG. 1 is a diagram showing the possible outcomes from mixing caseins and salts that lead to differences of structure and stability compared to native casein micelles.

FIG. 2 is an electropherogram from a capillary electrophoresis apparatus showing commercially sourced β-casein from Sigma Aldrich and in-house purified β-casein.

FIG. 3 is an electropherogram from a capillary electrophoresis apparatus showing commercially sourced κ-casein from Sigma Aldrich and in-house purified κ-casein.

FIG. 4 is an is an image of beta-casein micelle solutions made with increasing concentrations of kappa-casein.

FIG. 5 is a plot showing the turbidity of casein micelle solutions of beta-casein with increasing concentrations of kappa-casein, pre- and post-centrifugation.

FIG. 6 is a plot showing the hydrodynamic radius against particle concentration of the B8KX series measured by Nanoparticle tracking analysis.

FIG. 7 is an image of beta-casein micelles at different mineral concentrations versus a caseinate casein micelle control solution.

FIG. 8 is a plot showing the turbidity measurements of pre- and post-centrifugation of beta-casein only micelles.

FIG. 9 is a plot showing the hydrodynamic radius against particle concentration of the beta-casein only micelle samples 1, 2 and 3, measured by Nanoparticle tracking analysis.

FIG. 10 is a cryo-transmission electron micrograph of a beta-casein only casein micelle from sample 2.

FIG. 11 is an image of alpha-s-casein micelles at different mineral concentrations versus a caseinate casein micelle control solution.

FIG. 12 is a plot showing the turbidity of alpha-s-casein micelle solutions pre- and post-centrifugation.

FIG. 13 is a plot showing the hydrodynamic radius against particle concentration of the alpha-s-casein only micelle samples 1 and 2 measured by Nanoparticle tracking analysis.

### DETAILED DESCRIPTION

Before describing the present invention in detail, it is to be understood that the terminology used herein is for the purpose of describing embodiments only and is not intended to be limiting.

### Definitions

Unless specifically defined otherwise, all technical and scientific terms used herein shall be taken to have the same meaning as commonly understood by one of ordinary skill in the art.

As used in this specification and the appended claims, the singular forms “a”, “an” and “the” include plural referents unless the content clearly dictates otherwise.

Throughout the description and claims of the specification the word “comprise” and variations of the word, such as “comprising” and “comprises”, is not intended to exclude other additives, components, integers or steps. As used herein, “comprises” means “includes”. Thus, “comprising A or B,” means “including A, B, or A and B,” without excluding additional elements.

The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “a food product” refers to a substance that can be used or prepared for use as food, which is any nutritious substance that humans or animals eat or drink to maintain life and growth.

As used herein, the term “calcium sensitive casein” refers to casein proteins sensitive to precipitation by calcium ions. Similarly, the term “calcium insensitive casein” refers to casein proteins insensitive to precipitation by calcium ions. The calcium-sensitive bovine caseins are α_{S1}-, α_{S2}- and β-caseins whereas κ-casein is insensitive to precipitation by calcium ions. As used herein, the term “α_{S1}-casein” and “alpha-S1 casein” are used interchangeably and refers to all polymorphs of the CSN1S1 gene products encoding alpha-S1 casein. As used herein, the term “α_{S2}-casein” and “alpha-S2 casein” are used interchangeably and refers to all polymorphs of the CSN1S2 gene products for alpha-S2 casein. As used herein, the term “β-casein” and “beta casein” are used interchangeably and refers to all polymorphs of the CSN2 casein gene products encoding beta casein. As used herein, the term “κ-casein” and “kappa casein” are used interchangeably and refers to all polymorphs of the CSN3 gene products encoding kappa casein.

### Casein Micelle Compositions

The present invention relates to a casein micelle composition comprising casein micelle particles containing amorphous calcium phosphate (CaP), wherein the casein micelle particles comprise at least one calcium sensitive casein, and wherein the casein micelle particles do not contain κ-casein.

Casein micelles are colloidal particles formed by casein aggregates and are dispersed in liquid, forming a colloidal suspension or composition. Usually, the liquid is water but any suitable solvent can be used.

In different embodiments of the casein micelle composition of the present invention, the casein micelle particles comprise:
(i) only αₛ₁-casein;
(ii) only αₛ₂-casein;
(iii) only β-casein;
(iv) a mixture of αₛ₁- and β-casein;
(v) a mixture of αₛ₂- and β-casein;
(vi) a mixture of αₛ₁- and αₛ₂-casein; or
(vii) a mixture of αₛ₁-, αₛ₂- and β-casein.

In one embodiment, the casein micelle composition contains sufficient CaP to bind between about 5% and 100% of the casein. The casein micelle composition may contain sufficient CaP to bind at least 3% or about 5% of the casein. The casein micelle composition may contain sufficient CaP to bind at most 100% of the casein. The casein micelle composition may contain sufficient CaP to bind at least 5%, 10%, 15%, 20%, 25%, 30%, 35%, 40%, 45%, 50%, 55%, 60%, 65%, 70%, 75%, 80%, 85%, 90%, 95%, 100% of the casein.

In a further embodiment, the total casein concentration in the composition is between about 0.5-100 g L⁻¹. The total casein concentration in the composition may be at least about 0.5 g L⁻¹. The total casein concentration in the composition may be at most about 100 g L⁻¹. The total casein concentration in the composition may be 0.5, 1, 2, 3, 4, 5, 10, 15, 20, 25, 30, 35, 40, 45, 50, 55, 60, 65, 70, 75, 80, 85, 90, 95, or 100 g L⁻¹. The total casein concentration in the composition may be from about 0.5 to 5, 0.5 to 10, 0.5 to 15, 0.5 to 20, 0.5 to 25, 0.5 to 30, 0.5 to 35, 0.5 to 40, 0.5 to 45, 0.5 to 50, 0.5 to 55, 0.5 to 60, 0.5 to 65, 0.5 to 70, 0.5 to 75, 0.5 to 80, 0.5 to 85, 0.5 to 90, 0.5 to 95, 0.5 to 100, 1 to 5, 1 to 10, 1 to 15, 1 to 20, 1 to 25, 1 to 30, 1 to 35, 1 to 40, 1 to 45, 1 to 50, 1 to 55, 1 to 60, 1 to 65, 1 to 70, 1 to 75, 1 to 80, 1 to 85, 1 to 90, 1 to 95, 1 to 100, 2 to 5, 2 to 10, 2 to 15, 2 to 20, 2 to 25, 2 to 30, 2 to 35, 2 to 40, 2 to 45, 2 to 50, 2 to 55, 2 to 60, 2 to 65, 2 to 70, 2 to 75, 2 to 80, 2 to 85, 2 to 90, 2 to 95, 2 to 100, 3 to 5, 3 to 10, 3 to 15, 3 to 20, 3 to 25, 3 to 30, 3 to 35, 3 to 40, 3 to 45, 3 to 50, 3 to 55, 3 to 60, 3 to 65, 3 to 70, 3 to 75, 3 to 80, 3 to 85, 3 to 90, 3 to 95, or 3 to 100, 4 to 5, 4 to 10, 4 to 15, 4 to 20, 4 to 25, 4 to 30, 4 to 35, 4 to 40, 4 to 45, 4 to 50, 4 to 55, 4 to 60, 4 to 65, 4 to 70, 4 to 75, 4 to 80, 4 to 85, 4 to 90, 4 to 95, 4 to 100, 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 15 to 10, 15 to 15, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 40 to 45, 40 to 50, 40 to 55, 40 to 60, 40 to 65, 40 to 70, 40 to 75, 40 to 80, 40 to 85, 40 to 90, 40 to 95, 40 to 100, 45 to 50, 45 to 55, 45 to 60, 45 to 65, 45 to 70, 45 to 75, 45 to 80, 45 to 85, 45 to 90, 45 to 95, 45 to 100, 50 to 55, 50 to 60, 50 to 65, 50 to 70, 50 to 75, 50 to 80, 50 to 85, 50 to 90, 50 to 95, 50 to 100, 55 to 60, 55 to 65, 55 to 70, 55 to 75, 55 to 80, 55 to 85, 55 to 90, 55 to 95, 55 to 100, 60 to 65, 60 to 70, 60 to 75, 60 to 80, 60 to 85, 60 to 90, 60 to 95, 60 to 100, 65 to 70, 65 to 75, 65 to 80, 65 to 85, 65 to 90, 65 to 95, 65 to 100, 70 to 75, 70 to 80, 70 to 85, 70 to 90, 70 to 95, 70 to 100, 75 to 80, 75 to 85, 75 to 90, 75 to 95, 75 to 100, 80 to 85, 80 to 90, 80 to 95, 80 to 100, 85 to 90, 85 to 95, 85 to 100, 90 to 95, 90 to 100, or 95 to 100 g L⁻¹. Preferably, the total casein concentration in the composition may be about 5 to 50 g L⁻¹. More preferably, the total casein concentration in the composition may be about 30 g L⁻¹.

In another embodiment, the pH of the composition may be between about pH 5.5 and about pH 8.0. The pH of the composition may be at least pH 5.5 or about pH 6.0. The pH of the composition may be at most about pH 8.0. The pH of the composition may be about pH 5.5, pH 6.0, pH 6.5, pH 7.0, pH 7.5, or pH 8.0. The pH of the composition may be from about 5.5 to 6.0, 5.5 to 6.5, 5.5 to 7.0, 5.5 to 7.5, 5.5 to 8.0, 6.0 to 6.5, 6.0 to 7.0, 6.0 to 7.5, 6.0 to 8.0, 6.5 to 7.0, 6.5 to 7.5, 6.5 to 8.0, 7.0 to 7.5, 7.0 to 8.0, or 7.5 to 8.0. Preferably, the pH of the composition may be about pH 6.7.

In some embodiments, the casein micelle particles comprise a mixture of αₛ₁- and β-casein. In some embodiments, the casein micelle particles comprise a mixture of αₛ₂- and β-casein. In some embodiments, the casein micelle particles comprise a mixture of αₛ₁- and αₛ₂-casein.

In some embodiments, the casein micelle particles comprise a mixture of αₛ₁- and β-casein. The ratio of alpha S1 casein protein to beta casein protein in the casein micelle composition may be from about 1:15 to about 15:1. The ratio of alpha S1 casein protein to beta casein protein in the casein micelle composition may be about 15:1; 14:1; 13:1; 12:1; 11:1; 10:1; 9:1; 8:1; 7:1; 6:1; 5:1; 4:1; 3:1; 2:1; 1:1, 2:1, 3:1, 4:1, 5:1, 6:1, 7:1, 8:1, 9:1, 10:1, 11:1, 12:1, 13:1, 14:1 or 15:1.

In some embodiments, the casein micelle particles comprise a mixture of αₛ₂- and β-casein. The ratio of alpha S2 casein protein to beta casein protein in the casein micelle composition may be from about 1:15 to about 15:1. The ratio of alpha S2 casein protein to beta casein protein in the casein micelle composition may be about 15:1; 14:1; 13:1; 12:1; 11:1; 10:1; 9:1; 8:1; 7:1; 6:1; 5:1; 4:1; 3:1; 2:1; 1:1, 2:1, 3:1, 4:1, 5:1, 6:1, 7:1, 8:1, 9:1, 10:1, 11:1, 12:1, 13:1, 14:1 or 15:1.

In some embodiments, the casein micelle particles comprise a mixture of αₛ₁- and αₛ₂-casein. The ratio of alpha S1 casein protein to alpha S2 casein protein in the casein micelle composition may be from about 1:15 to about 15:1. The ratio of alpha S1 casein protein to alpha S2 casein protein in the casein micelle composition may be about 15:1; 14:1; 13:1; 12:1; 11:1; 10:1; 9:1; 8:1; 7:1; 6:1; 5:1; 4:1; 3:1; 2:1; 1:1, 2:1, 3:1, 4:1, 5:1, 6:1, 7:1, 8:1, 9:1, 10:1, 11:1, 12:1, 13:1, 14:1 or 15:1.

In the embodiments where the casein micelle particles comprise a mixture of two casein proteins (such as for example, a mixture of αₛ₁- and β-casein, or a mixture of αₛ₂- and β-casein, or a mixture of mixture of αₛ₁- and αₛ₂-casein), the ratio of one casein protein to another protein in the casein micelle composition may be from about 1:15 to about 15:1. For example, the ratio of one casein protein to another protein in the casein micelle composition may be from 1:15 to 15:1; 1:14 to 15:1; 1:13 to 15:1; 1:12 to 15:1; 1:11 to 15:1; 1:10 to 15:1; 1:9 to 15:1; 1:8 to 15:1; 1:7 to 15:1; 1:6 to 15:1; 1:5 to 15:1; 1:4 to 15:1; 1:3 to 15:1; 1:2 to 15:1; 1:15 to 15:1; 1:1 to 15:1; 2:1 to 15:1; 3:1 to 15:1; 4:1 to 15:1; 5:1 to 15:1; 6:1 to 15:1; 7:1 to 15:1; 8:1 to 15:1; 9:1 to 15:1; 10:1 to 15:1; 11:1 to 15:1; 12:1 to 15:1; 13:1 to 15:1; 14:1 to 15:1; 1:15 to 14:1; 1:14 to 14:1; 1:13 to 14:1; 1:12 to 14:1; 1:11 to 14:1; 1:10 to 14:1; 1:9 to 14:1; 1:8 to 14:1; 1:7 to 14:1; 1:6 to 14:1; 1:5 to 14:1; 1:4 to 14:1; 1:3 to 14:1; 1:2 to 14:1; 1:15 to 14:1; 1:1 to 14:1; 2:1 to 14:1; 3:1 to 14:1; 4:1 to 14:1; 5:1 to 14:1; 6:1 to 14:1; 7:1 to 14:1; 8:1 to 14:1; 9:1 to 14:1; 10:1 to 14:1; 11:1 to 14:1; 12:1 to 14:1; 13:1 to 14:1; 1:15 to 13:1; 1:14 to 13:1; 1:13 to 13:1; 1:12 to 13:1; 1:11 to 13:1; 1:10 to 13:1; 1:9 to 13:1; 1:8 to 13:1; 1:7 to 13:1; 1:6 to 13:1; 1:5 to 13:1; 1:4 to 13:1; 1:3 to 13:1; 1:2 to 13:1; 1:15 to 13:1; 1:1 to 13:1; 2:1 to 13:1; 3:1 to 13:1; 4:1 to 13:1; 5:1 to 13:1; 6:1 to 13:1; 7:1 to 13:1; 8:1 to 13:1; 9:1 to 13:1; 10:1 to 13:1; 11:1 to 13:1; 12:1 to 13:1; 1:15 to 12:1; 1:14 to 12:1; 1:13 to 12:1; 1:12 to 12:1; 1:11 to 12:1; 1:10 to 12:1; 1:9 to 12:1; 1:8 to 12:1; 1:7 to 12:1; 1:6 to 12:1; 1:5 to 12:1; 1:4 to 12:1; 1:3 to 12:1; 1:2 to 12:1; 1:15 to 12:1; 1:1 to 12:1; 2:1 to 12:1; 3:1 to 12:1; 4:1 to 12:1; 5:1 to 12:1; 6:1 to 12:1; 7:1 to 12:1; 8:1 to 12:1; 9:1 to 12:1; 10:1 to 12:1; 11:1 to 12:1; 1:15 to 11:1; 1:14 to 11:1; 1:13 to 11:1; 1:12 to 11:1; 1:11 to 11:1; 1:10 to 11:1; 1:9 to 11:1; 1:8 to 11:1; 1:7 to 11:1; 1:6 to 11:1; 1:5 to 11:1; 1:4 to 11:1; 1:3 to 11:1; 1:2 to 11:1; 1:15 to 11:1; 1:1 to 11:1; 2:1 to 11:1; 3:1 to 11:1; 4:1 to 11:1; 5:1 to 11:1; 6:1 to 11:1; 7:1 to 11:1; 8:1 to 11:1; 9:1 to 11:1; 10:1 to 11:1; 1:15 to 10:1; 1:14 to 10:1; 1:13 to 10:1; 1:12 to 10:1; 1:11 to 10:1; 1:10 to 10:1; 1:9 to 10:1; 1:8 to 10:1; 1:7 to 10:1; 1:6 to 10:1; 1:5 to 10:1; 1:4 to 10:1; 1:3 to 10:1; 1:2 to 10:1; 1:15 to 10:1; 1:1 to 10:1; 2:1 to 10:1; 3:1 to 10:1; 4:1 to 10:1; 5:1 to 10:1; 6:1 to 10:1; 7:1 to 10:1; 8:1 to 10:1; 9:1 to 10:1; 1:15 to 9:1; 1:14 to 9:1; 1:13 to 9:1; 1:12 to 9:1; 1:11 to 9:1; 1:10 to 9:1; 1:9 to 9:1; 1:8 to 9:1; 1:7 to 9:1; 1:6 to 9:1; 1:5 to 9:1; 1:4 to 9:1; 1:3 to 9:1; 1:2 to 9:1; 1:15 to 9:1; 1:1 to 9:1; 2:1 to 9:1; 3:1 to 9:1; 4:1 to 9:1; 5:1 to 9:1; 6:1 to 9:1; 7:1 to 9:1; 8:1 to 9:1; 1:15 to 8:1; 1:14 to 8:1; 1:13 to 8:1; 1:12 to 8:1; 1:11 to 8:1; 1:10 to 8:1; 1:9 to 8:1; 1:8 to 8:1; 1:7 to 8:1; 1:6 to 8:1; 1:5 to 8:1; 1:4 to 8:1; 1:3 to 8:1; 1:2 to 8:1; 1:15 to 8:1; 1:1 to 8:1; 2:1 to 8:1; 3:1 to 8:1; 4:1 to 8:1; 5:1 to 8:1; 6:1 to 8:1; 7:1 to 8:1; 1:15 to 7:1; 1:14 to 7:1; 1:13 to 7:1; 1:12 to 7:1; 1:11 to 7:1; 1:10 to 7:1; 1:9 to 7:1; 1:8 to 7:1; 1:7 to 7:1; 1:6 to 7:1; 1:5 to 7:1; 1:4 to 7:1; 1:3 to 7:1; 1:2 to 7:1; 1:15 to 7:1; 1:1 to 7:1; 2:1 to 7:1; 3:1 to 7:1; 4:1 to 7:1; 5:1 to 7:1; 6:1 to 7:1; 1:15 to 6:1; 1:14 to 6:1; 1:13 to 6:1; 1:12 to 6:1; 1:11 to 6:1; 1:10 to 6:1; 1:9 to 6:1; 1:8 to 6:1; 1:7 to 6:1; 1:6 to 6:1; 1:5 to 6:1; 1:4 to 6:1; 1:3 to 6:1; 1:2 to 6:1; 1:15 to 6:1; 1:1 to 6:1; 2:1 to 6:1; 3:1 to 6:1; 4:1 to 6:1; 5:1 to 6:1; 1:15 to 5:1; 1:14 to 5:1; 1:13 to 5:1; 1:12 to 5:1; 1:11 to 5:1; 1:10 to 5:1; 1:9 to 5:1; 1:8 to 5:1; 1:7 to 5:1; 1:6 to 5:1; 1:5 to 5:1; 1:4 to 5:1; 1:3 to 5:1; 1:2 to 5:1; 1:15 to 5:1; 1:1 to 5:1; 2:1 to 5:1; 3:1 to 5:1; 4:1 to 5:1; 1:15 to 4:1; 1:14 to 4:1; 1:13 to 4:1; 1:12 to 4:1; 1:11 to 4:1; 1:10 to 4:1; 1:9 to 4:1; 1:8 to 4:1; 1:7 to 4:1; 1:6 to 4:1; 1:5 to 4:1; 1:4 to 4:1; 1:3 to 4:1; 1:2 to 4:1; 1:15 to 4:1; 1:1 to 4:1; 2:1 to 4:1; 3:1 to 4:1; 1:15 to 3:1; 1:14 to 3:1; 1:13 to 3:1; 1:12 to 3:1; 1:11 to 3:1; 1:10 to 3:1; 1:9 to 3:1; 1:8 to 3:1; 1:7 to 3:1; 1:6 to 3:1; 1:5 to 3:1; 1:4 to 3:1; 1:3 to 3:1; 1:2 to 3:1; 1:15 to 3:1; 1:1 to 3:1; 2:1 to 3:1; 1:15 to 2:1; 1:14 to 2:1; 1:13 to 2:1; 1:12 to 2:1; 1:11 to 2:1; 1:10 to 2:1; 1:9 to 2:1; 1:8 to 2:1; 1:7 to 2:1; 1:6 to 2:1; 1:5 to 2:1; 1:4 to 2:1; 1:3 to 2:1; 1:2 to 2:1; 1:15 to 2:1; 1:1 to 2:1; 1:15 to 1:1; 1:14 to 1:1; 1:13 to 1:1; 1:12 to 1:1; 1:11 to 1:1; 1:10 to 1:1; 1:9 to 1:1; 1:8 to 1:1; 1:7 to 1:1; 1:6 to 1:1; 1:5 to 1:1; 1:4 to 1:1; 1:3 to 1:1; 1:2 to 1:1; 1:15 to 1:2; 1:14 to 1:2; 1:13 to 1:2; 1:12 to 1:2; 1:11 to 1:2; 1:10 to 1:2; 1:9 to 1:2; 1:8 to 1:2; 1:7 to 1:2; 1:6 to 1:2; 1:5 to 1:2; 1:4 to 1:2; 1:3 to 1:2; 1:15 to 1:3; 1:14 to 1:3; 1:13 to 1:3; 1:12 to 1:3; 1:11 to 1:3; 1:10 to 1:3; 1:9 to 1:3; 1:8 to 1:3; 1:7 to 1:3; 1:6 to 1:3; 1:5 to 1:3; 1:4 to 1:3; 1:15 to 1:4; 1:14 to 1:4; 1:13 to 1:4; 1:12 to 1:4; 1:11 to 1:4; 1:10 to 1:4; 1:9 to 1:4; 1:8 to 1:4; 1:7 to 1:4; 1:6 to 1:4; 1:5 to 1:4; 1:15 to 1:5; 1:14 to 1:5; 1:13 to 1:5; 1:12 to 1:5; 1:11 to 1:5; 1:10 to 1:5; 1:9 to 1:5; 1:8 to 1:5; 1:7 to 1:5; 1:6 to 1:5; 1:15 to 1:6; 1:14 to 1:6; 1:13 to 1:6; 1:12 to 1:6; 1:11 to 1:6; 1:10 to 1:6; 1:9 to 1:6; 1:8 to 1:6; 1:7 to 1:6; 1:15 to 1:7; 1:14 to 1:7; 1:13 to 1:7; 1:12 to 1:7; 1:11 to 1:7; 1:10 to 1:7; 1:9 to 1:7; 1:8 to 1:7; 1:15 to 1:8; 1:14 to 1:8; 1:13 to 1:8; 1:12 to 1:8; 1:11 to 1:8; 1:10 to 1:8; 1:9 to 1:8; 1:15 to 1:9; 1:14 to 1:9; 1:13 to 1:9; 1:12 to 1:9; 1:11 to 1:9; 1:10 to 1:9; 1:15 to 1:10; 1:14 to 1:10; 1:13 to 1:10; 1:12 to 1:10; 1:11 to 1:10; 1:15 to 1:11; 1:14 to 1:11; 1:13 to 1:11; 1:12 to 1:11; 1:15 to 1:12; 1:14 to 1:12; 1:13 to 1:12; 1:15 to 1:13; 1:14 to 1:13; or 1:15 to 1:14.

In some embodiments, the casein micelle particles comprise a mixture of αₛ₁-, αₛ₂- and β-casein. In some preferred embodiments, each of alpha S1 casein protein, alpha S2 casein protein, beta casein protein in the casein micelle composition may be present in an amount of between 1 and 15 parts. The amount of alpha S1 casein protein, alpha S2 casein protein and beta casein protein present in the casein micelle composition may be, in any order, from about 1:1:1, 1:1:2, 1:1:3, 1:1:4, 1:1:5, 1:1:6, 1:1:7, 1:1:8, 1:1:9, 1:1:10, 1:1:11, 1:1:12, 1:1:13, 1:1:14, 1:1:15, 1:2:2, 1:2:3, 1:2:4, 1:2:5, 1:2:6, 1:2:7, 1:2:8, 1:2:9, 1:2:10, 1:2:11, 1:2:12, 1:2:13, 1:2:14, 1:2:15, 1:3:3, 1:3:4, 1:3:5, 1:3:6, 1:3:7, 1:3:8, 1:3:9, 1:3:10, 1:3:11, 1:3:12, 1:3:13, 1:3:14, 1:3:15, 1:4:4, 1:4:5, 1:4:6, 1:4:7, 1:4:8, 1:4:9, 1:4:10, 1:4:11, 1:4:12, 1:4:13, 1:4:14, 1:4:15, 1:5:5, 1:5:6, 1:5:7, 1:5:8, 1:5:9, 1:5:10, 1:5:11, 1:5:12, 1:5:13, 1:5:14, 1:5:15, 1:6:6, 1:6:7, 1:6:8, 1:6:9, 1:6:10, 1:6:11, 1:6:12, 1:6:13, 1:6:14, 1:6:15, 1:7:7, 1:7:8, 1:7:9, 1:7:10, 1:7:11, 1:7:12, 1:7:13, 1:7:14, 1:7:15, 1:8:8, 1:8:9, 1:8:10, 1:8:11, 1:8:12, 1:8:13, 1:8:14, 1:8:15, 1:9:9, 1:9:10, 1:9:11, 1:9:12, 1:9:13, 1:9:14, 1:9:15, 1:10:10, 1:10:11, 1:10:12, 1:10:13, 1:10:14, 1:10:15, 1:11:11, 1:11:12, 1:11:13, 1:11:14, 1:11:15, 1:12:12, 1:12:13, 1:12:14, 1:12:15, 1:13:13, 1:13:14, 1:13:15, 1:14:14, 1:14:15, 1:15:15, 2:2:3, 2:2:4, 2:2:5, 2:2:6, 2:2:7, 2:2:8, 2:2:9, 2:2:10, 2:2:11, 2:2:12, 2:2:13, 2:2:14, 2:2:15, 2:3:3, 2:3:4, 2:3:5, 2:3:6, 2:3:7, 2:3:8, 2:3:9, 2:3:10, 2:3:11, 2:3:12, 2:3:13, 2:3:14, 2:3:15, 2:4:4, 2:4:5, 2:4:6, 2:4:7, 2:4:8, 2:4:9, 2:4:10, 2:4:11, 2:4:12, 2:4:13, 2:4:14, 2:4:15, 2:5:5, 2:5:6, 2:5:7, 2:5:8, 2:5:9, 2:5:10, 2:5:11, 2:5:12, 2:5:13, 2:5:14, 2:5:15, 2:6:6, 2:6:7, 2:6:8, 2:6:9, 2:6:10, 2:6:11, 2:6:12, 2:6:13, 2:6:14, 2:6:15, 2:7:7, 2:7:8, 2:7:9, 2:7:10, 2:7:11, 2:7:12, 2:7:13, 2:7:14, 2:7:15, 2:8:8, 2:8:9, 2:8:10, 2:8:11, 2:8:12, 2:8:13, 2:8:14, 2:8:15, 2:9:9, 2:9:10, 2:9:11, 2:9:12, 2:9:13, 2:9:14, 2:9:15, 2:10:10, 2:10:11, 2:10:12, 2:10:13, 2:10:14, 2:10:15, 2:11:11, 2:11:12, 2:11:13, 2:11:14, 2:11:15, 2:12:12, 2:12:13, 2:12:14, 2:12:15, 2:13:13, 2:13:14, 2:13:15, 2:14:14, 2:14:15, 2:15:15, 3:3:4, 3:3:5, 3:3:6, 3:3:7, 3:3:8, 3:3:9, 3:3:10, 3:3:11, 3:3:12, 3:3:13, 3:3:14, 3:3:15, 3:4:4, 3:4:5, 3:4:6, 3:4:7, 3:4:8, 3:4:9, 3:4:10, 3:4:11, 3:4:12, 3:4:13, 3:4:14, 3:4:15, 3:5:5, 3:5:6, 3:5:7, 3:5:8, 3:5:9, 3:5:10, 3:5:11, 3:5:12, 3:5:13, 3:5:14, 3:5:15, 3:6:6, 3:6:7, 3:6:8, 3:6:9, 3:6:10, 3:6:11, 3:6:12, 3:6:13, 3:6:14, 3:6:15, 3:7:7, 3:7:8, 3:7:9, 3:7:10, 3:7:11, 3:7:12, 3:7:13, 3:7:14, 3:7:15, 3:8:8, 3:8:9, 3:8:10, 3:8:11, 3:8:12, 3:8:13, 3:8:14, 3:8:15, 3:9:9, 3:9:10, 3:9:11, 3:9:12, 3:9:13, 3:9:14, 3:9:15, 3:10:10, 3:10:11, 3:10:12, 3:10:13, 3:10:14, 3:10:15, 3:11:11, 3:11:12, 3:11:13, 3:11:14, 3:11:15, 3:12:12, 3:12:13, 3:12:14, 3:12:15, 3:13:13, 3:13:14, 3:13:15, 3:14:14, 3:14:15, 3:15:15, 4:4:5, 4:4:6, 4:4:7, 4:4:8, 4:4:9, 4:4:10, 4:4:11, 4:4:12, 4:4:13, 4:4:14, 4:4:15, 4:5:5, 4:5:6, 4:5:7, 4:5:8, 4:5:9, 4:5:10, 4:5:11, 4:5:12, 4:5:13, 4:5:14, 4:5:15, 4:6:6, 4:6:7, 4:6:8, 4:6:9, 4:6:10, 4:6:11, 4:6:12, 4:6:13, 4:6:14, 4:6:15, 4:7:7, 4:7:8, 4:7:9, 4:7:10, 4:7:11, 4:7:12, 4:7:13, 4:7:14, 4:7:15, 4:8:8, 4:8:9, 4:8:10, 4:8:11, 4:8:12, 4:8:13, 4:8:14, 4:8:15, 4:9:9, 4:9:10, 4:9:11, 4:9:12, 4:9:13, 4:9:14, 4:9:15, 4:10:10, 4:10:11, 4:10:12, 4:10:13, 4:10:14, 4:10:15, 4:11:11, 4:11:12, 4:11:13, 4:11:14, 4:11:15, 4:12:12, 4:12:13, 4:12:14, 4:12:15, 4:13:13, 4:13:14, 4:13:15, 4:14:14, 4:14:15, 4:15:15, 5:5:6, 5:5:7, 5:5:8, 5:5:9, 5:5:10, 5:5:11, 5:5:12, 5:5:13, 5:5:14, 5:5:15, 5:6:6, 5:6:7, 5:6:8, 5:6:9, 5:6:10, 5:6:11, 5:6:12, 5:6:13, 5:6:14, 5:6:15, 5:7:7, 5:7:8, 5:7:9, 5:7:10, 5:7:11, 5:7:12, 5:7:13, 5:7:14, 5:7:15, 5:8:8, 5:8:9, 5:8:10, 5:8:11, 5:8:12, 5:8:13, 5:8:14, 5:8:15, 5:9:9, 5:9:10, 5:9:11, 5:9:12, 5:9:13, 5:9:14, 5:9:15, 5:10:10, 5:10:11, 5:10:12, 5:10:13, 5:10:14, 5:10:15, 5:11:11, 5:11:12, 5:11:13, 5:11:14, 5:11:15, 5:12:12, 5:12:13, 5:12:14, 5:12:15, 5:13:13, 5:13:14, 5:13:15, 5:14:14, 5:14:15, 5:15:15, 6:6:7, 6:6:8, 6:6:9, 6:6:10, 6:6:11, 6:6:12, 6:6:13, 6:6:14, 6:6:15, 6:7:7, 6:7:8, 6:7:9, 6:7:10, 6:7:11, 6:7:12, 6:7:13, 6:7:14, 6:7:15, 6:8:8, 6:8:9, 6:8:10, 6:8:11, 6:8:12, 6:8:13, 6:8:14, 6:8:15, 6:9:9, 6:9:10, 6:9:11, 6:9:12, 6:9:13, 6:9:14, 6:9:15, 6:10:10, 6:10:11, 6:10:12, 6:10:13, 6:10:14, 6:10:15, 6:11:11, 6:11:12, 6:11:13, 6:11:14, 6:11:15, 6:12:12, 6:12:13, 6:12:14, 6:12:15, 6:13:13, 6:13:14, 6:13:15, 6:14:14, 6:14:15, 6:15:15, 7:7:8, 7:7:9, 7:7:10, 7:7:11, 7:7:12, 7:7:13, 7:7:14, 7:7:15, 7:8:8, 7:8:9, 7:8:10, 7:8:11, 7:8:12, 7:8:13, 7:8:14, 7:8:15, 7:9:9, 7:9:10, 7:9:11, 7:9:12, 7:9:13, 7:9:14, 7:9:15, 7:10:10, 7:10:11, 7:10:12, 7:10:13, 7:10:14, 7:10:15, 7:11:11, 7:11:12, 7:11:13, 7:11:14, 7:11:15, 7:12:12, 7:12:13, 7:12:14, 7:12:15, 7:13:13, 7:13:14, 7:13:15, 7:14:14, 7:14:15, 7:15:15, 8:8:9, 8:8:10, 8:8:11, 8:8:12, 8:8:13, 8:8:14, 8:8:15, 8:9:9, 8:9:10, 8:9:11, 8:9:12, 8:9:13, 8:9:14, 8:9:15, 8:10:10, 8:10:11, 8:10:12, 8:10:13, 8:10:14, 8:10:15, 8:11:11, 8:11:12, 8:11:13, 8:11:14, 8:11:15, 8:12:12, 8:12:13, 8:12:14, 8:12:15, 8:13:13, 8:13:14, 8:13:15, 8:14:14, 8:14:15, 8:15:15, 9:9:10, 9:9:11, 9:9:12, 9:9:13, 9:9:14, 9:9:15, 9:10:10, 9:10:11, 9:10:12, 9:10:13, 9:10:14, 9:10:15, 9:11:11, 9:11:12, 9:11:13, 9:11:14, 9:11:15, 9:12:12, 9:12:13, 9:12:14, 9:12:15, 9:13:13, 9:13:14, 9:13:15, 9:14:14, 9:14:15, 9:15:15, 10:10:11, 10:10:12, 10:10:13, 10:10:14, 10:10:15, 10:11:11, 10:11:12, 10:11:13, 10:11:14, 10:11:15, 10:12:12, 10:12:13, 10:12:14, 10:12:15, 10:13:13, 10:13:14, 10:13:15, 10:14:14, 10:14:15, 10:15:15, 11:11:12, 11:11:13, 11:11:14, 11:11:15, 11:12:12, 11:12:13, 11:12:14, 11:12:15, 11:13:13, 11:13:14, 11:13:15, 11:14:14, 11:14:15, 11:15:15, 12:12:13, 12:12:14, 12:12:15, 12:13:13, 12:13:14, 12:13:15, 12:14:14, 12:14:15, 12:15:15, 13:13:14, 13:13:15, 13:14:14, 13:14:15, 13:15:15, 14:14:15, 14:15:15, or 15:15:15.

Micelle diameters, such as micelles in the casein micelles of the present invention, herein may be from about 10 nm to about 500 nm. Micelle diameters herein may be at least 10 nm. Micelle diameters herein may be at most 500 nm. Micelle diameters herein may be from 10 nm to 20 nm, 10 nm to 50 nm, 10 nm to 100 nm, 10 nm to 150 nm, 10 nm to 200 nm, 10 nm to 250 nm, 10 nm to 300 nm, 10 nm to 350 nm, 10 nm to 400 nm, 10 nm to 450 nm, 10 nm to 500 nm, 20 nm to 50 nm, 20 nm to 100 nm, 20 nm to 150 nm, 20 nm to 200 nm, 20 nm to 250 nm, 20 nm to 300 nm, 20 nm to 350 nm, 20 nm to 400 nm, 20 nm to 450 nm, 20 nm to 500 nm, 50 nm to 100 nm, 50 nm to 150 nm, 50 nm to 200 nm, 50 nm to 250 nm, 50 nm to 300 nm, 50 nm to 350 nm, 50 nm to 400 nm, 50 nm to 450 nm, 50 nm to 500 nm, 100 nm to 150 nm, 100 nm to 200 nm, 100 nm to 250 nm, 100 nm to 300 nm, 100 nm to 350 nm, 100 nm to 400 nm, 100 nm to 450 nm, 100 nm to 500 nm, 150 nm to 200 nm, 150 nm to 250 nm, 150 nm to 300 nm, 150 nm to 350 nm, 150 nm to 400 nm, 150 nm to 450 nm, 150 nm to 500 nm, 200 nm to 250 nm, 200 nm to 300 nm, 200 nm to 350 nm, 200 nm to 400 nm, 200 nm to 450 nm, 200 nm to 500 nm, 250 nm to 300 nm, 250 nm to 350 nm, 250 nm to 400 nm, 250 nm to 450 nm, 250 nm to 500 nm, 300 nm to 350 nm, 300 nm to 400 nm, 300 nm to 450 nm, 300 nm to 500 nm, 350 nm to 400 nm, 350 nm to 450 nm, 350 nm to 500 nm, 400 nm to 450 nm, 400 nm to 500 nm, or 450 nm to 500 nm. Micelle diameters herein may be about 10 nm, about 20 nm, about 50 nm, about 100 nm, about 150 nm, about 200 nm, about 250 nm, about 300 nm, about 350 nm, about 400 nm, about 450 nm, or about 500 nm. Micelle diameters herein may be at least 10 nm, 20 nm, 50 nm, 100 nm, 150 nm, 200 nm, 250 nm, 300 nm, 350 nm, 400 nm or 450 nm. Micelle diameters herein may be at most 20 nm, 50 nm, 100 nm, 150 nm, 200 nm, 250 nm, 300 nm, 350 nm, 400 nm, 450 nm or 500 nm.

In another embodiment, the casein micelle composition comprises (i) only αₛ₁-casein, and the concentration of calcium in the composition is between 1.48 and 150.00 mM, preferably between 2.72 and 38.58 mM. The concentration of calcium in the composition may be at least about 1.48 mM. The concentration of calcium in the composition may be at most about 150.00 mM. The concentration of calcium in the composition may be about 1.48, 1.85, 2.20, 2.55, 2.93, 3.30, 3.70, 4.1, 4.50, 4.80, 7.70, 14.30, 20.90, 27.50, 34.15, 40.8, 47.30, 54.00, 60.60, 67.20, 70.0, 75.0, 80.0, 85.0, 90.0, 95.0, 100.0, 105.0, 110.0, 115.0, 120.0, 125.0, 130.0, 135.0, 140.0, 145.0, or 150.0 mM. Preferably, the concentration of calcium in the composition may be between 1.48 and 150.00 mM, more preferably between 2.72 and 38.58 mM.

In another embodiment, the casein micelle composition comprises (i) only αₛ₁-casein, and the concentration of phosphate in the composition is between 23.5 and 58.40 mM, preferably between 24.42 and 43.66 mM. The concentration of phosphate in the composition may be at least 23.5 mM. The concentration of phosphate in the composition may be at most about 58.5 mM. The concentration of phosphate in the composition may be about 23.30, 23.20, 23.00, 22.80, 22.60, 22.30, 22.20, 22.10, 22.00, 28.10, 31.80, 35.50, 39.2, 42.90, 46.60, 49.00, 52.00, 55.10, or 58.40 mM. Preferably, the concentration of phosphate in the composition may be between 23.5 and 58.40 mM, more preferably between 24.42 and 43.66 mM.

In another embodiment, the casein micelle composition comprises (i) only αₛ₂-casein, and the concentration of calcium in the composition is between 1.48 and 150 mM, preferably between 2.72 and 38.58 mM. The concentration of calcium in the composition may be at least about 1.48 mM. The concentration of calcium in the composition may be at most about 150.00 mM. The concentration of calcium in the composition may be about 1.48, 1.85, 2.20, 2.55, 2.93, 3.30, 3.70, 4.1, 4.50, 4.80, 7.70, 14.30, 20.90, 27.50, 34.15, 40.8, 47.30, 54.00, 60.60, 67.20, 70.0, 75.0, 80.0, 85.0, 90.0, 95.0, 100.0, 105.0, 110.0, 115.0, 120.0, 125.0, 130.0, 135.0, 140.0, 145.0, or 150.0 mM. Preferably, the concentration of calcium in the composition may be between 1.48 and 150 mM, preferably between 2.72 and 38.58 mM.

In another embodiment, the casein micelle composition comprises (i) only αₛ₂-casein, and the concentration of phosphate in the composition is between 23.5 and 58.40 mM, preferably between 24.42 and 43.66 mM. The concentration of phosphate in the composition may be at least 23.5 mM. The concentration of phosphate in the composition may be at most about 58.4 mM. The concentration of phosphate in the composition may be about 23.5, 23.3, 23.2, 23.0, 22.8, 22.6, 22.3, 22.2, 22.1, 22.0, 28.1, 31.8, 35.5, 39.21, 42.9, 46.6, 49.0, 51.5, 54.7, or 58.40 mM. Preferably, the concentration of phosphate in the composition may be between 23.5 and 58.40 mM, more preferably between 24.42 and 43.66 mM.

In another embodiment, the casein micelle composition comprises (i) only β-casein, and the concentration of calcium in the composition is between 1.3 and 34.0 mM, preferably between 1.81 and 7.69 mM. The concentration of calcium in the composition may be at least about 1.3 mM. The concentration of calcium in the composition may be at most about 35.00 mM. The concentration of calcium in the composition may be about 1.3, 1.5, 1.85, 2.2, 2.20, 2.55, 2.93, 3.30, 3.70, 4.1, 4.35, 4.50, 4.80, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 32.0, 33.0, 34.0, or 35.0 mM. Preferably, the concentration of calcium in the composition may be between 1.3 and 34.0 mM, more preferably between 1.81 and 7.69 mM.

In another embodiment, the casein micelle composition comprises (i) only β-casein, and the concentration of phosphate in the composition is between 24.54 and 41.0 mM, preferably between 24.54 and 36.1 mM. The concentration of phosphate in the composition may be at least 24.0 mM. The concentration of phosphate in the composition may be at most about 45.0 mM. The concentration of phosphate in the composition may be about 24.0, 25.0, 26.0, 27.0, 28.0, 29.0, 30.0, 31.0, 32.0, 33.0, 32.0, 33.0, 34.0, 35.0, 36.0, 37.0, 38.0, 39.0, 40.0, 41.0, 42.0, 43.0, 44.0, or 45.0 mM. Preferably, the concentration of phosphate in the composition may be between 24.54 and 41.0 mM, more preferably between 24.54 and 36.1 mM.

In yet another embodiment, the composition comprises a mixture of calcium sensitive caseins; and the calcium and phosphate concentrations are a combination of the ranges for the individual casein components but combined in proportion to the mole fraction of each casein in the mixture.

In some embodiments, the casein micelle compositions of the present invention have shown a stability that is comparable to naturally occurring micelle compositions. Ideally, the casein micelle particles remain dispersed in their colloidal suspension or composition under comparable conditions to naturally occurring micelle compositions. For both artificial and naturally occurring micelle compositions, under certain conditions, the dispersion of casein micelles will separate, and casein will precipitate.

In some embodiments, the casein micelle compositions of the present invention remain dispersed under centrifugation at 4000×g for 3 mins. In some embodiments, the casein micelle compositions of the present invention remain dispersed under centrifugation at 3000×g for 5 mins.

In some further embodiments, the casein micelle compositions remain dispersed when pasteurised for 15 seconds at 72° C. In some further embodiments, the casein micelle compositions remain dispersed when pasteurised for 1-2 seconds at 135° C.

In one embodiment of the present invention, the calcium sensitive casein protein is recombinantly produced. A recombinant casein protein is recombinantly expressed in a host cell. As used herein, a “host” or “host cell” denotes any protein production host selected or genetically modified to produce a desired product. Exemplary hosts include fungi, bacteria, yeast, plant, insect, and mammalian cells. Preferably, the casein proteins of the present invention are recombinantly produced from a host cell that is selected from the group consisting of bacteria, yeast, and fungi.

When the host cell is a bacteria, the bacterial host cell is preferably selected from the group consisting of Lactococci sp., Lactococcus lactis, Bacillus subtilis, Bacillus amyloliquefaciens, Bacillus licheniformis and Bacillus megaterium, Brevibacillus chohinensis, Mycobacterium smegmatic, Rhodococcus erythroplois and Corynebacterium glutamicum, Lactobacilli sp., Lactobacillus fermentum, Lactobacillus casei, Lactobacillus acidophilus, Lactobacillus plantarum, Synechocystis sp. 6803 and E. coli. Preferably, the bacterial host cell is Lactococcus lactis, Bacillus subtilis, or E. coli.

When the host cell is a yeast, the yeast host cell is preferably selected from the group consisting of Kluyveromyces sp., Pichia sp., Saccharomyces sp., Tetrahynena sp., Yarrowia sp., Hansenula sp., Blastobotrys sp., Candida sp., Zygosaccharomyces sp., or Debaryomyces sp.

When the host cell is a fungus, the fungal host cell is preferably selected from the group consisting of any Aspergillus sp. (such as Aspergillus nidulans, Aspergillus niger, Aspergillus oryzae), Trichoderma reesei, Chrysosporium lucknowense, Fusarium sp., Fusarium granineum, Fusarium venenatum, Physcoinitrella patens, and Neurospora crassa.

In some embodiments, the casein protein comprises an amino acid sequence from any species. Particularly the casein protein comprises an amino acid sequence from any eutherian, metatherian or monotreme species that commonly contribute to the human diet such as, for example, bovine, ovine, caprine, cameline, equine or primate species. In some preferred embodiment, the casein protein comprises an amino acid sequence selected from the group consisting of cow, goat, sheep, buffalo, camel, horse, bison, human, donkey, chimpanzee, rabbit, mouse, guinea pig, brush-trailed possum, duckbill platypus, Australian echidna, wallaby, zebu, or mixtures thereof.

The calcium sensitive casein proteins of the present invention may be produced in the same host cell. Alternatively, the calcium sensitive casein proteins may be produced in different host cells.

Depending on the host cell used to express the casein protein, the casein proteins may have a glycosylation or phosphorylation pattern (post-translational modifications) different from animal-derived casein proteins. In some cases, the casein protein comprises no post translational modifications (PTMs). In some cases, the casein protein comprises substantially reduced PTMs. As used herein, substantially reduced PTMs means at least 50% reduction of one or more types of PTMs as compared to the amount of PTMs in an animal-derived casein protein. For instance, casein proteins may be 50%, 55%, 60%, 65%, 70%, 75%, 80%, 85%, 90%, 92%, 95%, 97%, 99% less post-translationally modified as compared to animal-derived casein protein. Alternatively, the casein protein may comprise PTMs comparable to animal-derived casein PTMs.

The PTMs in the casein protein may be modified chemically or enzymatically. In some cases, the casein protein comprises substantially reduced or no PTMs without chemical or enzymatic treatment. Casein micelles may be generated using casein protein with reduced or no PTMs, wherein the lack of or reduction of PTMs is not due to chemical or enzymatic treatments, such as by producing recombinant casein protein in a host where the casein protein is not post-translationally modified or the level of PTMs is substantially reduced.

The glycosylation in the casein protein may be modified chemically or enzymatically. In some cases, the casein protein comprises substantially reduced or no glycosylation without chemical or enzymatic treatment. For instance, casein proteins may be 50%, 55%, 60%, 65%, 70%, 75%, 80%, 85%, 90%, 92%, 95%, 97%, 99% less glycosylated as compared to animal-derived casein. Casein micelles may be generated using casein protein with reduced or no glycosylation, wherein the lack of glycosylation is not due to chemical or enzymatic treatments post recombinant production.

The phosphorylation in the casein protein may be modified chemically or enzymatically. In some cases, the casein protein comprises substantially reduced or no phosphorylation without chemical or enzymatic treatment. For instance, casein proteins may be 50%, 55%, 60%, 65%, 70%, 75%, 80%, 85%, 90%, 92%, 95%, 97%, 99% less phosphorylated as compared to animal-derived casein. Casein micelles may be generated using casein protein with reduced or no phosphorylation, wherein the lack of phosphorylation is not due to chemical or enzymatic treatments, such as by producing recombinant protein in a host where the casein protein is not post-translationally modified or the level of PTMs is substantially reduced.

The casein micelle compositions of the present invention do not comprise kappa casein. The proteins of the present invention therefore do not comprise a protein sequence for kappa casein (SEQ ID NOs: 50-66).

In preferred embodiments, the recombinantly produced casein protein has an amino acid sequence comprising SEQ ID NO. 1-49 (as shown in Table 1) or a variant thereof with at least 80% sequence homology. The protein may have at least 80%, 85%, 90%, 95%, 96%, 97%, 98%, or 99% sequence identity to SEQ ID NO. 1-49 (as shown in Table 1).

TABLE 1
Protein amino acid sequences containing signal peptides shown in bold. Mature
protein is without the signal peptide.

| SEQ | | | | Accession |
|---|---|---|---|---|
| ID No. | Protein | Species | Sequence | number |
| 1 | as1-casein | Bovine | MKLLILTCLVAVALARPKHPIKHQGLPQEVLN | P02662 |
| | | | ENLLRFFVAPFPEVFGKEKVNELSKDIGSESTED | |
| | | | QAMEDIKQMEAESISSSEEIVPNSVEQKHIQKE | |
| | | | DVPSERYLGYLEQLLRLKKYKVPQLEIVPNSAEE | |
| | | | RLHSMKEGIHAQQKEPMIGVNQELAYFYPELF | |
| | | | RQFYQLDAYPSGAWYYVPLGTQYTDAPSFSDI | |
| | | | PNPIGSENSEKTTMPLW | |
| 2 | as1-casein | Domestic | MKLLILTCLVAVALARPKQPIKHQGLPQGVLN | O62823 |
| | | water buffalo | ENLLRFFVAPFPEVFGKEKVNELSTDIGSESTED | |
| | | | QAMEDIKQMEAESISSSEEIVPISVEQKHIQKE | |
| | | | DVPSERYLGYLEQLLRLKKYNVPQLEIVPNLAEE | |
| | | | QLHSMKEGIHAQQKEPMIGVNQELAYFYPQL | |
| | | | FRQFYQLDAYPSGAWYYVPLGTQYPDAPSFS | |
| | | | DIPNPIGSENSGKTTMPLW | |
| 3 | as1-casein | Goat | MKLLILTCLVAVALARPKHPINHRGLSPEVPNE | P18626 |
| | | | NLLRFVVAPFPEVFRKENINELSKDIGSESTEDQ | |
| | | | AMEDAKQMKAGSSSSSEEIVPNSAEQKYIQKE | |
| | | | DVPSERYLGYLEQLLRLKKYNVPQLEIVPKSAEE | |
| | | | QLHSMKEGNPAHQKQPMIAVNQELAYFYPQ | |
| | | | LFRQFYQLDAYPSGAWYYLPLGTQYTDAPSFS | |
| | | | DIPNPIGSENSGKTTMPLW | |
| 4 | as1-casein | Sheep | MKLLILTCLVAVALARPKHPIKHQGLSSEVLNE | P04653 |
| | | | NLLRFVVAPFPEVFRKENINELSKDIGSESIEDQ | |
| | | | AMEDAKQMKAGSSSSSEEIVPNSAEQKYIQKE | |
| | | | DVPSERYLGYLEQLLRLKKYNVPQLEIVPKSAEE | |
| | | | QLHSMKEGNPAHQKQPMIAVNQELAYFYPQ | |
| | | | LFRQFYQLDAYPSGAWYYLPLGTQYTDAPSFS | |
| | | | DIPNPIGSENSGKITMPLW | |
| 5 | as1-casein | Dog | MKFLILTCLVAVALARPKLPLRHPELTQNELDS | J9P7W6 |
| | | | REPTPRELREEYFSELSKRELLREKQNEGIKVQV | |
| | | | MEDPEQRQSSSTSSSEEVVPNNTEEQLRRLSQ | |
| | | | HNQLQVGTIHDQQQLRRVNENNLLQLPFQQF | |
| | | | YQLDAYPYAAWYFPAQIMQYIAYPPSLDITKPI | |
| | | | ASENIENADVVPQW | |
| 6 | as1-casein | Pig | MKLLIFICLAAVALARPKPPLRHQEHLQNEPD | P39035 |
| | | | SREELFKERKFLRFPEVPLLSQFRQEIINELNRN | |
| | | | HGMEGHEQRGSSSSSSEEVVGNSAEQKHVQK | |
| | | | EEDVPSQSYLGHLQGLNKYKLRQLEAIHDQEL | |
| | | | HRTNEDKHTQQGEPMKGVNQEQAYFYFEPL | |
| | | | HQFYQLDAYPYATWYYPPQYIAHPLFTNIPQP | |
| | | | TAPEKGGKTEIMPQW | |
| 7 | as1-casein | Chimpanzee | MRLLILTCLVAVALARPKLPLRYPERLQNPSES | H2QPK8 |
| | | | SEPIPLESRQEYMNGMNRQRNILREKQTDEIK | |
| | | | DTRNESTQNCVMAEPEKMESSISSSSEEISLSK | |
| | | | CAEQFCRLNEYNQLQLQAVHAQEQIRRMNE | |
| | | | NSHVQVPFQQLNQLAAYPCAVWYYPQIMQY | |
| | | | VPFPPFSDISNPTAHENYEKNNVMLQW | |
| 8 | as1-casein | Dromedary | MKLLILTCLVAVALARPKYPLRYPEVFQNEPDS | O97943 |
| | | | IEEVLNKRKILELAVVSPIQFRQENIDELKDTRN | |
| | | | EPTEDHIMEDTERKESGSSSSEEVVSSTTEQKDI | |
| | | | LKEDMPSQRYLEELHRLNKYKLLQLEAIRDQKLI | |
| | | | PRVKLSSHPYLEQLYRINEDNHPQLGEPVKVVT | |
| | | | QEQAYFHLEPFPQFFQLGASPYVAWYYPPQV | |
| | | | MQYIAHPSSYDTPEGIASEDGGKTDVMPQW | |
| | | | W | |
| 9 | as1-casein | Equus asinus | MKLLILTCLVAVALARPKLPHRHPEIIQNEQDS | C3W972 |
| | | africanu | REKVLKERKFPSFALEYINELNRQRELLKEKQKD | |
| | | | EHKEYLIEDPEQQESSSTSSSEEVVPINTEQKRI | |
| | | | PREDMLYQHTLEALRRLSKYNQLQLQAIYAQE | |
| | | | QLLRMKENSQRKPMRVVNQEQAYFYLEPFQP | |
| | | | SYQLDVYPYAAWFHPAQIMQHVAYSPFHDTA | |
| | | | KLIASENSEKTDIIPEW | |
| 10 | as1-casein | Human | MRLLILTCLVAVALARPKLPLRYPERLQNPSES | P47710 |
| | | | SEPIPLESREEYMNGMNRQRNILREKQTDEIK | |
| | | | DTRNESTQNCVVAEPEKMESSISSSSEEMSLSK | |
| | | | CAEQFCRLNEYNQLQLQAAHAQEQIRRMNE | |
| | | | NSHVQVPFQQLNQLAAYPYAVWYYPQIMQY | |
| | | | VPFPPFSDISNPTAHENYEKNNVMLQW | |
| 11 | as1-casein | Brush-tailed | MKLLIFSCLMALALARPDVLHLSIDRHIKHREV | Q9XSE3 |
| | | possum | ENRSNEDLIPLNEVSSSEESLHQLNRDRRSPEK | |
| | | | YELNKYREDLKTSSSEESVAPSTEESVRRQVEY | |
| | | | NFNEQEDASASRERKIEDVSEQYRQYLRRRPE | |
| | | | ERALNLRYLEPLYYATEPDFYYTYVPISMPRFFP | |
| | | | YPAEAPVFSTRKAPVPSINRATEAVYTYSEEKK | |
| 12 | as1-casein | Duckbill | MKVLILACLVAVAVAMPESPSSSSSSEEAPRLL | D0QJ96 |
| | | platypus | TKKRILRNQEYYLPHLEESRSSSSSEESTRPTLKR | |
| | | | LLLKEKPILHILKAPESSSSEESDSAAEKRLLRERE | |
| | | | FYQQQLDEYYRQFEPDFYPRAYPKKEVMPYPL | |
| | | | EYFIPQAAVYSIPQLVYRVPQEVTFPSPLRFRYA | |
| | | | FPQPTLPVERK | |
| 13 | as1-casein | Australian | MKVLILACLVAFVVAMPESPSSSSSSEEASKILT | D0QJA2 |
| | | echidna | KKRVQRDQEYYLPHQEESVSSSSSEESTDRLKR | |
| | | | RLLKDKPIFRLLKATESSSSEESDSAIEKRILRERQ | |
| | | | YYQQKLDELKEYFRQFEPYFYPVAYQKKEVMP | |
| | | | YQLEYFVPQPEVYSIPQPVYRVPQEVTFPSLLH | |
| | | | FRYAFPQSTLPIERK | |
| 14 | as1-casein | Rabbit | MKLLILTCLVATALARHKFHLGHLKLTQEQPES | P09115 |
| | | | SEQEILKERKLLRFVQTVPLELREEYVNELNRQR | |
| | | | ELLREKENEEIKGTRNEVTEEHVLADRETEASIS | |
| | | | SSSEEIVPSSTKQKYVPREDLAYQPYVQQQLLR | |
| | | | MKERYQIQEREPMRVVNQELAQLYLQPFEQP | |
| | | | YQLDAYLPAPWYYTPEVMQYVLSPLFYDLVTP | |
| | | | SAFESAEKTDVIPEWLKN | |
| 15 | as1-casein | Mouse | MKLLILTCLVAAAFAMPRLHSRNAVSSQTQQ | P19228 |
| | | | QHSSSEEIFKQPKYLNLNQEFVNNMNRQRALL | |
| | | | TEQNDEIKVTMDAASEEQAMASAQEDSSISSS | |
| | | | SEESEEAIPNITEQKNIANEDMLNQCTLEQLQR | |
| | | | QFKYNQLLQKASLAKQASLFQQPSLVQQASLF | |
| | | | QQPSLLQQASLFQQPSMAQQASLLQQLLLAQ | |
| | | | QPSLALQVSPAQQSSLVQQAFLAQQASLAQK | |
| | | | HHPRLSQSYYPHMEQPYRMNAYSQVQMRH | |
| | | | PMSVVDQALAQFSVQPFPQIFQYDAFPLWAY | |
| | | | FPQDMQYLTPKAVLNTFKPIVSKDTEKTNVW | |
| 16 | as1-casein | Guinea pig | MKLLILTCLVASAVAMPKFPFRHTELFQTQRG | P04656 |
| | | | GSSSSSSSEERLKEENIFKFDQQKELQRKQSEKI | |
| | | | KEIISESTEQREASSISSSEEVVPKNTEQKHIPQE | |
| | | | DALYQQALEQLSRLIKYHQLQMEVVHAQEQF | |
| | | | HRINEHNQAQVKEPMRVFNQLDAYPFAAWY | |
| | | | YGPEVQYMSFLPFSSIPQPIFPEDAQNTEVMP | |
| | | | EWVM | |
| 17 | as2-casein | Bovine | MKFFIFTCLLAVALAKNTMEHVSSSEESIISQET | P02663 |
| | | | YKQEKNMAINPSKENLCSTFCKEVVRNANEEE | |
| | | | YSIGSSSEESAEVATEEVKITVDDKHYQKALNEI | |
| | | | NQFYQKFPQYLQYLYQGPIVLNPWDQVKRNA | |
| | | | VPITPTLNREQLSTSEENSKKTVDMESTEVFTK | |
| | | | KTKLTEEEKNRLNFLKKISQRYQKFALPQYLKTV | |
| 18 | as2-casein | Domestic | MKFFIFTCLLAVALAKHTMEHVSSSEESIISQET | B6VPY2 |
| | | water buffalo | YKQEKNMAIHPSKENLCSTFCKEVIRNANEEEY | |
| | | | SIGSSSEESAEVATEEVKITVDDKHYQKALNEIN | |
| | | | QFYQKFPQYLQYLYQGPIVLNPWDQVKRNAV | |
| | | | PITPTLNREQLSTSEENSKKTVDMESTEVITKKT | |
| | | | KLTEEDKNRLNFLKKISQHYQKFTWPQYLKTVY | |
| | | | QYQKAMKPWTQPKTNVIPYVRYL | |
| 19 | as2-casein | Goat | MKFFIFTCLLAVALAKHKMEHVSSSEEPINIFQ | P33049 |
| | | | EIYKQEKNMAIHPRKEKLCTTSCEEVVRNANEE | |
| | | | EYSIRSSSEESAEVAPEEIKITVDDKHYQKALNEI | |
| | | | NQFYQKFPQYLQYPYQGPIVLNPWDQVKRNA | |
| | | | GPFTPTVNREQLSTSEENSKKTIDMESTEVFTK | |
| | | | KTKLTEEEKNRLNFLKKISQYYQKFAWPQYLKT | |
| | | | VDQHQKAMKPWTQPKTNAIPYVRYL | |
| 20 | as2-casein | Sheep | MKFFIFTCLLAVALAKHKMEHVSSSEEPINISQ | P04654 |
| | | | EIYKQEKNMAIHPRKEKLCTTSCEEVVRNADEE | |
| | | | EYSIRSSSEESAEVAPEEVKITVDDKHYQKALNE | |
| | | | INQFYQKFPQYLQYLYQGPIVLNPWDQVKRN | |
| | | | AGPFTPTVNREQLSTSEENSKKTIDMESTEVFT | |
| | | | KKTKLTEEEKNRLNFLKKISQYYQKFAWPQYLK | |
| | | | TVDQHQKAMKPWTQPKTNAIPYVRYL | |
| 21 | as2-casein | Pig | MKFFIFTCLLAVAFAKHEMEHVSSSEESINISQ | P39036 |
| | | | EKYKQEKNVINHPSKEDICATSCEEAVRNIKEV | |
| | | | GYASSSSSEESVDIPAENVKVTVEDKHYLKQLE | |
| | | | KISQFYQKFPQYLQALYQAQIVMNPWDQTKT | |
| | | | SAYPFIPTVIQSGEELSTSEEPVSSSQEENTKTV | |
| | | | DMESMEEFTKKTELTEEEKNRIKFLNKIKQYYQ | |
| | | | KFTWPQYIKTVHQKQKAMKPWNHIKTNSYQI | |
| | | | IPNLRYF | |
| 22 | as2-casein | Dromedary | MKFFIFTCLLAVVLAKHEMDQGSSSEESINVS | O97944 |
| | | | QQKFKQVKKVAIHPSKEDICSTFCEEAVRNIKE | |
| | | | VESAEVPTENKISQFYQKWKFLQYLQALHQG | |
| | | | QIVMNPWDQGKTRAYPFIPTVNTEQLSISEES | |
| | | | TEVPTEESTEVFTKKTELTEEEKDHQKFLNKIYQ | |
| | | | YYQTFLWPEYLKTVYQYQKTMTPWNHIKRYF | |
| 23 | as2-casein | Donkey | MKFFIFTCLLAVALAKHNMEHRSSSEDSVNIS | B7VGF9 |
| | | | QEKFKQEKYVVIPTSKESICSTSCEEATRNINEM | |
| | | | ESAKFPTEVYSSSSSSEESAKFPTEREEKEVEEK | |
| | | | HHLKQLNKINQFYEKLNFLQYLQALRQPRIVLT | |
| | | | PWDQTKTGASPFIPIVNTEQLFTSEEIPKKTVD | |
| | | | MESTEVVTEKTELTEEEKNYLKLLNKINQYYEKF | |
| | | | TLPQYFKIVHQHQTTMDPQSHSKTNSYQIIPVL | |
| | | | RYF | |
| 24 | as2-casein | Guinea pig | MKLFIFTCLLAVALAKHKSEQQSSSEESVSISQE | P04655 |
| | | | KFKDKNMDTISSEETICASLCKEATKNTPKMAF | |
| | | | FSRSSSEEFADIHRENKKDQLYQKWMVPQYN | |
| | | | PDFYQRPVVMSPWNQIYTRPYPIVLPTLGKEQ | |
| | | | ISTIEDILKKTTAVESSSSSSTEKSTDVFIKKTKM | |
| | | | DEVQKLIQSLLNIIHEYSQKAFWSQTLEDVDQY | |
| | | | LKFVMPWNHYNTNADQVDASQERQA | |
| 25 | as2-casein | Rabbit | MKFFIFTCLLAVALAKPKIEQSSSEETIAVSQEV | P50419 |
| | | | SPNLENICSTACEEPIKNINEVEYVEVPTEIKDQ | |
| | | | EFYQKVNLLQYLQALYQYPTVMDPWTRAETK | |
| | | | AIPFIRTMQYKQEKDATKHTSQKTELTEEEKAF | |
| | | | LKYLDEMKQYYQKFVFPQYLKNAHHFQKTMN | |
| | | | PWNHVKTIIYQSVPTLRYL | |
| 26 | as2-casein | Mouse | MKFFIFACLVVVALAKHEIKDKSSSEESSASIYP | Q02862 |
| | | | GKSKLDNSVFFQTTKDSASSSSSEESSEEVSEKI | |
| | | | VQSEEQKVNLNQQKKFKQFSQESSFSQCCTPL | |
| | | | HQQQQSSVNQWPQPNAIHNTPTQESISTSVE | |
| | | | EILKKIIDMIKYIQYQQVTIPQLPQALHPQIPVSY | |
| | | | WYPSKDYTFPNAHYTRFY | |
| 27 | as2-casein | Rat | MKFFIFTCLVAAALAKHAVKDKPSSEESASVYL | P02667 |
| | | | GKYKQGNSVFFQTPQDSASSSSSEESSEEISEKI | |
| | | | EQSEEQKVNLNQQKKSKQFSQDSSFPQICTPY | |
| | | | QQQSSVNQRPQPNAIYDVPSQESTSTSVEEILK | |
| | | | KIIDIVKYFQYQQLTNPHFPQAVHPQIRVSSW | |
| | | | APSKDYTFPTARYMA | |
| 28 | as2-casein | Rabbit | MRFFVFTCLLAVALAKNGIEQRSASEEIVSFYQ | P50418 |
| | | | EKYKQDSNAAIYPTNQETPSVSSSEESVEVQTE | |
| | | | KDEQIEEENVYLKQLKRIKQIFQKFYIPQYPEVY | |
| | | | QQQIVMNPWKHVKTTTYPVPIPETTRIPLEEIV | |
| | | | KKIVEMIKFNQLHQFVIPQYVQALQQRIAMNP | |
| | | | WHHVTPFRSFPVLNF | |
| 29 | as2-casein | Mouse | MKFIILTCLLAVALAKQRMEQYISSEESMDNS | P02664 |
| | | | QENFKQNMDVAFFPSQETVENIYIPQMESVE | |
| | | | APMKVSDIISQQQYNQKMMDMSVSAREKTV | |
| | | | MTEESKNIQDYMNKMKRYSKITWPQFVKLLH | |
| | | | QYQKTMTPWSYYPSTPSQV | |
| 30 | as2-casein | Rat | MKFIILTCLLAVALAKQESKDNSQEDFKQTVD | Q8CGR3 |
| | | | VVIFPGQETVKNIPIPQMESVEAPIKNKCYQSI | |
| | | | QTFKPPQALKGLYQYHMAKNPWGYTVNRAF | |
| | | | PSTRTLQYNQKTMDLSMRAREKIVMSEIKKNI | |
| | | | QDYVTKMKQYSKITWPRFVKSLQQYQKTMN | |
| | | | PWSCYPYTLLQV | |
| 31 | as2-casein | Duckbill | MKVFILACLVAAAVAVPVSTEFDKLLVKEKLLK | D0QJ98 |
| | | platypus | HRDLVKDLPTIFSSEWEQFLRHPEVYVPLEKYQ | |
| | | | QRLRLFKPTYLVPVNKFVERHPFRNILFPEELPE | |
| | | | AYQPIEKEDSSSSSEETVQVPVEKHLLRLRKLHV | |
| | | | PQKLRPLRFYPNHQVPFXRHPLPYAGTQVHQ | |
| | | | PVEVPFPLPXQY | |
| 32 | as2-casein | Australian | MKVFIFACLVAVAMAVPKQQSSSSSSEETDK | D0QJA6 |
| | | echidna | QLVMENLLKHRALVKDIPTTFSSEENINYEKQ | |
| | | | WEQLLRQPMVYEPFEIYQQGLRPFKPTHLRRP | |
| | | | LKYIFFSEEPPKVYQPIQNEDSSSSSEEPVEVPA | |
| | | | EQNHVLRLKKLQVLQNLQPLRRLPNYQVPLQR | |
| | | | HPLPFVRLPNVFQAPHPVELPFPLPQVV | |
| 33 | beta-casein | Bovine | MKVLILACLVALALARELEELNVPGEIVESLSSS | P02666 |
| | | | EESITRINKKIEKFQSEEQQQTEDELQDKIHPFA | |
| | | | QTQSLVYPFPGPIPNSLPQNIPPLTQTPVVVPP | |
| | | | FLQPEVMGVSKVKEAMAPKHKEMPFPKYPVE | |
| | | | PFTESQSLTLTDVENLHLPLPLLQSWMHQPHQ | |
| | | | PLPPTVMFPPQSVLSLSQSKVLPVPQKAVPYP | |
| | | | QRDMPIQAFLLYQEPVLGPVRGPFPIIV | |
| 34 | beta-casein | Domestic | MKVLILACLVALALARELEELNVPGEIVESLSSS | Q9TSI0 |
| | | water buffalo | EESITHINKKIEKFQSEEQQQMEDELQDKIHPF | |
| | | | AQTQSLVYPFPGPIPKSLPQNIPPLTQTPVVVP | |
| | | | PFLQPEIMGVSKVKEAMAPKHKEMPFPKYPV | |
| | | | EPFTESQSLTLTDVENLHLPLPLLQSWMHQPP | |
| | | | QPLPPTVMFPPQSVLSLSQSKVLPVPQKAVPY | |
| | | | PQRDMPIQAFLLYQEPVLGPVRGPFPIIV | |
| 35 | beta-casein | Goat | MKVLILACLVALAIAREQEELNVVGETVESLSS | P33048 |
| | | | SEESITHINKKIEKFQSEEQQQTEDELQDKIHPF | |
| | | | AQAQSLVYPFTGPIPNSLPQNILPLTQTPVVVP | |
| | | | PFLQPEIMGVPKVKETMVPKHKEMPFPKYPV | |
| | | | EPFTESQSLTLTDVEKLHLPLPLVQSWMHQPP | |
| | | | QPLSPTVMFPPQSVLSLSQPKVLPVPQKAVPQ | |
| | | | RDMPIQAFLLYQEPVLGPVRGPFPILV | |
| 36 | beta-casein | Sheep | MKVLILACLVALALAREQEELNVVGETVESLSS | P11839 |
| | | | SEESITHINKKIEKFQSEEQQQTEDELQDKIHPF | |
| | | | AQAQSLVYPFTGPIPNSLPQNILPLTQTPVVVP | |
| | | | PFLQPEIMGVPKVKETMVPKHKEMPFPKYPV | |
| | | | EPFTESQSLTLTDVEKLHLPLPLVQSWMHQPP | |
| | | | QPLPPTVMFPPQSVLSLSQPKVLPVPQKAVPQ | |
| | | | RDMPIQAFLLYQEPVLGPVRGPFPILV | |
| 37 | beta-casein | Pig | MKLLILACFVALALARAKEELNASGETVESLSS | P39037 |
| | | | SEESITHISKEKIEKLKREEQQQTENERQNKIHQ | |
| | | | FPQPQPLAHPYTEPIPYPILPQNILPLAQVPVVV | |
| | | | PLLHPEVMKDSKAKETIVPKRKGMPFPKSPAE | |
| | | | PFVEGQSLTLTDFEVLSLPLLQSLMHQIPQPVP | |
| | | | QTPMFAPQPLLSLPQAKVLPVPQQVVPFPQR | |
| | | | DMPFQALLLYQDPLLGPLQGFYPVPQPVAPVY | |
| | | | NPV | |
| 38 | beta-casein | Dromedary | MKVLILACRVALALAREKEEFKTAGEALESISSS | Q9TVD0 |
| | | | EESITHINKQKIEKFKIEEQQQTEDEQQDKIYTF | |
| | | | PQPQSLVYSHTEPIPYPILPQNFLPPLQPAVMV | |
| | | | PFLQPKVMDVPKTKETIIPKRKEMPLLQSPVVP | |
| | | | FTESQSLTLTDLENLHLPLPLLQSLMYQIPQPVP | |
| | | | QTPMIPPQSLLSLSQFKVLPVPQQMVPYPQR | |
| | | | AMPVQAVLPFQEPVPDPVRGLHPVPQPLVPV | |
| | | | IA | |
| 39 | beta-casein | Horse | MKILILACLVALALAREKEELNVSSETVESLSSN | Q9GKK3 |
| | | | EPDSSSEESITHINKEKLQKFKHEGQQQREVER | |
| | | | QDKISRFVQPQPVVYPYAEPVPYAVVPQSILPL | |
| | | | AQPPILPFLQPEIMEVSQAKETILPKRKVMPFL | |
| | | | KSPIVPFSERQILNPTNGENLRLPVHLIQPFMH | |
| | | | QVPQSLLQTLMLPSQPVLSPPQSKVAPFPQPV | |
| | | | VPYPQRDTPVQAFLLYQDPRLGPTGELDPATQ | |
| | | | PIVAVHNPVIV | |
| 40 | beta-casein | Dog | MKVFILACLVALALAREKEELTLSNETVESLSSS | Q9N2G8 |
| | | | EESITHINKQKLENFKHEEQQQREDERQNKIH | |
| | | | PLFQQQPLVSPYADPIHYAILPQNILPLAQPAV | |
| | | | VVPFLQPEIMEVPKVKENIFPRHKVMPFLKSPV | |
| | | | TPFLDSQILNVADLENVHFPLPLSLPLLQPLMH | |
| | | | QIPQPLPLLQPLMHQIPQPLPQTPMLTPQSVL | |
| | | | SIPQPKVLPFPQQVVPYLQRDMPLQAFLPYQE | |
| | | | STHQAQPVTQPLAPLVNSALV | |
| 41 | beta-casein | Human | MKVLILACLVALALARETIESLSSSEESITEYKQK | P05814 |
| | | | VEKVKHEDQQQGEDEHQDKIYPSFQPQPLIYP | |
| | | | FVEPIPYGFLPQNILPLAQPAVVLPVPQPEIME | |
| | | | VPKAKDTVYTKGRVMPVLKSPTIPFFDPQIPKL | |
| | | | TDLENLHLPLPLLQPLMQQVPQPIPQTLALPP | |
| | | | QPLWSVPQPKVLPIPQQVVPYPQRAVPVQAL | |
| | | | LLNQELLLNPTHQIYPVTQPLAPVHNPISV | |
| 42 | beta-casein | Rabbit | MKVLILACLVALALAREKEQLSVPTEAVGSVSS | P09116 |
| | | | SEEITHINKQKLETIKHVEQLLREEKLQDKILPFI | |
| | | | QSLFPFAERIPYPTLPQNILNLAQLDMLLPLLQP | |
| | | | EIMEDPKAKETIIPKHKLMPFLKSPKTVPFVDS | |
| | | | QILNLREMKNQHLLLPQLLPFMHQVFQPFPQ | |
| | | | TPIPYPQALLSLPQSKFMPIVPQVVPYPQRDM | |
| | | | PIQALQLFQELLFPTHQGYPVVQPIAPVNV | |
| 43 | beta-casein | Rat | MKVFILACLVALALAREKDAFTVSSETGSISSEE | P02665 |
| | | | SVEHINEKLQKVKLMGQVQSEDVLQNKFHSGI | |
| | | | QSEPQAIPYAQTISCSPIPQNIQPIAQPPVVPTV | |
| | | | GPIISPELESFLKAKATVLPKHKQMPFLNSETVL | |
| | | | RLFNSQIPSLDLANLHLPQSPAQLQAQIVQAFP | |
| | | | QTPAVVSSQPQLSHPQSKSQYLVQQLAPLFQ | |
| | | | QGMPVQDLLQYLDLLLNPTLQFLATQQLHSTS | |
| | | | V | |
| 44 | beta-casein | Mouse | MKVFILACLVALALARETTFTVSSETDSISSEES | P10598 |
| | | | VEHINEQKLQKVNLMGQLQAEDVLQAKVHSS | |
| | | | IQSQPQAFPYAQAQTISCNPVPQNIQPIAQPP | |
| | | | VVPSLGPVISPELESFLKAKATILPKHKQMPLLN | |
| | | | SETVLRLINSQIPSLASLANLHLPQSLVQLLAQV | |
| | | | VQAFPQTHLVSSQTQLSLPQSKVLYFLQQVAP | |
| | | | FLPQDMSVQDLLQYLELLNPTVQFPATPQHSV | |
| | | | SV | |
| 45 | beta-casein | African | MKVFILACLVAFALGREKEEIIVSTETVENLSSS | G3U197 |
| | | elephant | EIRQFYSEESVTQVNKQKPEGVKHEEQQREDE | |
| | | | HQNKIQPLFQPQPLVYPFAEPIPYTVFPPNAIPL | |
| | | | AQPIVVLPFPQPEVKQLPEAKEITFPRQKLMSF | |
| | | | LKSPVMPFFDPQIPNLGTDLENLHLPLPLLQPL | |
| | | | RHQLHQPLAQTPVLPLPLSLPKVLPVPQQVIPY | |
| | | | PQRGRPIQNLQLYEEPLLDPTRKIYPVAQPLAP | |
| | | | VYNPVAV | |
| 46 | beta-casein | Brush-tailed | MKLLILTCLVVLAVARPMVEKISESEEHVTDVP | Q9XSE4 |
| | | possum | ENEHRLEINRYLRPEYEMMNLYYQPFYWSEE | |
| | | | MRNLKMTSLPKDRRMAVLKSVVSDDMLPPL | |
| | | | QHKSLSLPKPKVLPLSHRQILPPHTLRMVPLSH | |
| | | | KLFTIPKREMLPISERERLPAHERENLLAHEREIL | |
| | | | LAPQREMSLIPEREILLAAERVVLPEQEREIRPD | |
| | | | NEREVLAVHKREILPASEKEKVLPLFQERVLPLH | |
| | | | RREIVPPYQRDTIARREILPVDQRELMPEVVAV | |
| | | | DLYPFFQPVANFYYPAELNEKN | |
| 47 | beta-casein | Duckbill | MKVFILSCLLAVAMAMPKLQSSSSSSEETDQL | D0QJ95 |
| | | platypus | LVKEKLVKRRELMDLPTTLSSEEHVMEEKEFYQ | |
| | | | PRLKYPYPFFPPIKTYVNPHIYQKPAVLPVTHPE | |
| | | | TLTYLQPQQNPEDMPLPKKEVLPYLKAVVVPY | |
| | | | PQVQVMPYPETEVMPYFPPMTMSLVQPDIV | |
| | | | PPSFYREAVIRPVAYNLPPTVQKIPETN | |
| 48 | beta-casein | Australian | MKVFILACLVAVAMALPKQHSSSSSSEESDRL | D0QJ99 |
| | | echidna | LVKDIPTAFSSEEHSVDPKELYEPRQSYSYPWQ | |
| | | | SVRPINTYTYPRAYQIPAVLPMTHPQTLTYLQP | |
| | | | QFKPEDMSISQKQIPPYVQAVVMPYPQVEAIP | |
| | | | FPGAEFMPYAQPITTPLLQPEVFSAPFYREAVF | |
| | | | KPVIYGLPQSQPVQKIPETD | |
| 49 | beta-casein | Australian | MKVFIFACLVAVAMAVPKQQSSSSSSEETDK | D0QJA4 |
| | | echidna | QLVMENLLKHRALVENINYEKQWEQLLRQPM | |
| | | | VYEPFERRPLKYIFFSEEPPKVYQPIQNEDSSSSS | |
| | | | EEPVEVPAEQNHVLRLKKLQVLQNLQPLRRLP | |
| | | | NYQVPLQRHPLPFVRLPNVFQAPHPVELPFPL | |
| | | | PQVV | |
| 50 | kappa- | Bovine | MMKSFFLVVTILALTLPFLGAQEQNQEQPIRC | P02668 |
| | casein | | EKDERFFSDKIAKYIPIQYVLSRYPSYGLNYYQQ | |
| | | | KPVALINNQFLPYPYYAKPAAVRSPAQILQWQ | |
| | | | VLSNTVPAKSCQAQPTTMARHPHPHLSFMAI | |
| | | | PPKKNQDKTEIPTINTIASGEPTSTPTTEAVEST | |
| | | | VATLEDSPEVIESPPEINTVQVTSTAV | |
| 51 | kappa- | Domestic | MMKSFFLVVTILALTLPFLGAQEQNQEQPIRC | P11840 |
| | casein | water buffalo | EKEERFFNDKIAKYIPIQYVLSRYPSYGLNYYQQ | |
| | | | KPVALINNQFLPYPYYAKPAAVRSPAQILQWQ | |
| | | | VLPNTVPAKSCQAQPTTMTRHPHPHLSFMAI | |
| | | | PPKKNQDKTEIPTINTIVSVEPTSTPTTEAIENTV | |
| | | | ATLEASSEVIESVPETNTAQVTSTVV | |
| 52 | kappa- | Sheep | MMKSFFLVVTILALTLPFLGAQEQNQEQRICC | P02669 |
| | casein | | EKDERFFDDKIAKYIPIQYVLSRYPSYGLNYYQQ | |
| | | | RPVALINNQFLPYPYYAKPVAVRSPAQTLQW | |
| | | | QVLPNAVPAKSCQDQPTAMARHPHPHLSFM | |
| | | | AIPPKKDQDKTEIPAINTIASAEPTVHSTPTTEA | |
| | | | VVNAVDNPEASSESIASAPETNTAQVTSTEV | |
| 53 | kappa- | Goat | MMKSFFLVVTILALTLPFLGAQEQNQEQPICC | P02670 |
| | casein | | EKDERFFDDKIAKYIPIQYVLSRYPSYGLNYYQQ | |
| | | | RPVALINNQFLPYPYYAKPVAVRSPAQTLQW | |
| | | | QVLPNTVPAKSCQDQPTTLARHPHPHLSFMAI | |
| | | | PPKKDQDKTEVPAINTIASAEPTVHSTPTTEAIV | |
| | | | NTVDNPEASSESIASASETNTAQVTSTEV | |
| 54 | kappa- | Dromedary | MKSFFLVVTILALTLPFLGAEVQNQEQPTCFEK | P79139 |
| | casein | | VERLLNEKTVKYFPIQFVQSRYPSYGINYYQHR | |
| | | | LAVPINNQFIPYPNYAKPVAIRLHAQIPQCQAL | |
| | | | PNIDPPTVERRPRPRPSFIAIPPKKTQDKTVNP | |
| | | | AINTVATVEPPVIPTAEPAVNTVVIAEASSEFIT | |
| | | | TSTPETTTVQITSTEI | |
| 55 | kappa- | Pig | MMKSSFLIVPILALTLPFLGAEEQNQEKLTRCE | P11841 |
| | casein | | SDKRLFNEEKVKYIPIYYMLNRFPSYGFFYQHR | |
| | | | SAVSPNRQFIPYPYYARPVVAGPHAQKPQWQ | |
| | | | DQPNVYPPTVARRPRPHASFIAIPPKKNQDKT | |
| | | | AIPAINSIATVEPTIVPATEPIVNAEPIVNAVVTP | |
| | | | EASSEFLITSAPETTTVQVTSPVV | |
| 56 | kappa- | Rabbit | MMKHFLLVVNILAVTLPFLAADIQNQEQTTC | P33618 |
| | casein | | RENEERLFHQVTAPYIPVHYVMNRYPQYEPSY | |
| | | | YLRRQAVPTLNPFMLNPYYVKPIVFKPNVQVP | |
| | | | HWQILPNIHQPKVGRHSHPFFMAILPNKMQD | |
| | | | KAVTPTTNTIAAVEPTPIPTTEPVVSTEVIAEAS | |
| | | | PELIISPETTTEATAASAAA | |
| 57 | kappa- | Guinea pig | MMKSFLLVVNIVALTLPFLAAEVQNQEQPAC | P19442 |
| | casein | | CGNDERLFEQKKVLYLLSYPVLNNYLRTAPSYY | |
| | | | QNRASVPINNPYLCHLYYVPSFVLWAQGQIPK | |
| | | | GPVSTDIHQSTMQYHQAKHPSFMAILSKKILG | |
| | | | KATILSTDAIAAPEQTPVSAAQPTVSAGDTPEV | |
| | | | SSQFIDTPDTSVLAEEARESPEDTPEISEFINAP | |
| | | | DTAVPSEEPRESAEDTPEISSEFIFSPETSTGPAI | |
| | | | ASMA | |
| 58 | kappa- | Rat | MMRNFIVVMNILALTLPFLAAEVQNPDSNCR | P04468 |
| | casein | | EKNEVVYDVQRVLYTPVSSVLNRNHYEPIYYHY | |
| | | | RTSVPVSPYAYFPVGLKLLLLRSPAQILKWQPM | |
| | | | PNFPQPVGVPHPIPNPSFLAIPTNEKHDNTAIP | |
| | | | ASNTIAPIVSTPVSTTESVVNTVANTEASTVPIS | |
| | | | TPETATVPVTSPAA | |
| 59 | kappa- | Mouse | MMRNFIVVVNILALTLPFLAAEIQNPDSNCRG | P06796 |
| | casein | | EKNDIVYDEQRVLYTPVRSVLNFNQYEPNYYH | |
| | | | YRPSLPATASPYMYYPLVVRLLLLRSPAPISKW | |
| | | | QSMPNFPQSAGVPYAIPNPSFLAMPTNENQD | |
| | | | NTAIPTIDPITPIVSTPVPTMESIVNTVANPEAS | |
| | | | TVSINTPETTTVPVSSTAA | |
| 60 | kappa- | Human | MKSFLLVVNALALTLPFLAVEVQNQKQPACH | P07498 |
| | casein | | ENDERPFYQKTAPYVPMYYVPNSYPYYGTNLY | |
| | | | QRRPAIAINNPYVPRTYYANPAVVRPHAQIPQ | |
| | | | RQYLPNSHPPTVVRRPNLHPSFIAIPPKKIQDKI | |
| | | | IIPTINTIATVEPTPAPATEPTVDSVVTPEAFSESI | |
| | | | ITSTPETTTVAVTPPTA | |
| 61 | kappa- | Horse | MKSFFLVVNILALTLPFLGAEVQNQEQPTCHK | P82187 |
| | casein | | NDERFFDLKTVKYIPIYYVLNSSPRYEPIYYQHRL | |
| | | | ALLINNQHMPYQYYARPAAVRPHVQIPQWQ | |
| | | | VLPNIYPSTVVRHPCPHPSFIAIPPKKLQEITVIP | |
| | | | KINTIATVEPTPIPTPEPTVNNAVIPDASSEFIIAS | |
| | | | TPETTTVPVTSPVVQKL | |
| 62 | kappa- | Dog | MMKRFFLVVNIVALALPFLQGAEVQNQEQP | E2QXF8 |
| | casein | | TCRENDERLFNQKTVKYIPIHYVLNSFSHYEPN | |
| | | | YYPHRPAEPINHQYVPYPFYAKPAVAVRTHAQ | |
| | | | IPQWQVLPNAYPPTMMHRPQLHPSFIAIPPKK | |
| | | | IQDKTSIPTINTIATAEATPIPLTEPKVNTAVTSD | |
| | | | ASSEFTITSTPETTTVPVTSPVV | |
| 63 | kappa- | African | MMKGFLLVVNILLLPLPFLAAEVQNQEESRSC | G3UDT9 |
| | casein | elephant | CLEKDERWFCQKAVKYIPNDYVLKSYYRYEPN | |
| | | | YNQFRAAVPINNPYLIYLYPAKQVAVRPHTQIP | |
| | | | QWQVPSNIYPSPSVPHTYLKPPFIVIPPKKTQD | |
| | | | KPIIPPTGTVASIEATVEPKVNTVVNAEASSEFI | |
| | | | ATNTPEATTVPVISPQI | |
| 64 | kappa- | Duckbill | MKTLLLVGAILAMTVGFSVAEEQKWKRLDSS | D0QJA9 |
| | casein | platypus | ESEERWWRLRLKPSLLFRVQDKPERNIPRPSYP | |
| | | | YPLLNVPHPNAINPEHQRPYVLPRFNFQIPNIL | |
| | | | PFLMFPELPPPFFPIVHPIYYDPQTPTTPRNPPV | |
| | | | TSQTPQPPVDSSANTPEPPTTAPLTATPEAQT | |
| | | | PLQP | |
| 65 | kappa- | Australian | MKTLLLVGGILVMTVCFSAAEDEEWKKVDYS | D0QJA7 |
| | casein | echidna | ESEERWLRLKRQPSFPFSFQGKPERNIPRPYYP | |
| | | | RPFLNIPRPYTINPEHQFAYVFPNLKFQIPSVFP | |
| | | | FPLEFLPPFYPFVHPIYYGPQTSTPPRNPTVTSQ | |
| | | | TPQPPVHSSANTPESATAAPVTATPMAQTPL | |
| | | | QP | |
| 66 | kappa- | Brush-tailed | MKVLFLTVHILAVMVCFSTADLDWEKWPCD | Q9XSD6 |
| | casein | possum | KQNERQSELRQQPLRRSPVQYVYTPYTHQSYV | |
| | | | PVIYPPRAYVRHPYFSRVAWQKPYPSYMPLLP | |
| | | | SIYPWSVVSRNLHPAFAFNPPHYAQLPVPSSP | |
| | | | TNSPTTTIQTTNIPITNPTSTIVTPAVSSKSAATE | |
| | | | DSAAAAMLTSPTAAQMA | |

In some embodiments of the present invention, the casein micelle compositions further comprise one or more non-casein proteins. This additional one or more proteins may be any suitable protein. Preferably, the non-casein protein is selected from the group consisting of osteopontin, dentin matrix protein, matrix extracellular phosphoglycoprotein, bone sialoprotein, dentin sialophosphoprotein, amelogenin, statherin, starmaker or otolith matrix macromolecule-64 and related homologs, calcium-binding proteins, secretory calcium-binding phosphoproteins (SCPPs) and mixtures thereof.

### Methods of Making Casein Micelle Compositions

The present invention also relates to a method of producing a casein micelle composition as disclosed herein, said method comprising combining at least one calcium sensitive casein protein with at least one salt under conditions wherein the at least one calcium sensitive casein protein forms a casein micelle particle in a liquid colloid, wherein the casein micelle particle does not include κ-casein protein.

Preferably, the at least one salt is a calcium salt and/or a phosphate salt. More preferably, at least two salts are used and these are at least one salt calcium salt and at least one phosphate salt.

In some embodiments, the method further comprises the addition of at least one further salt. Any suitable salt may be used, including calcium, phosphate, citrate, potassium, sodium and/or chloride salts. The calcium salt may be selected from calcium chloride, calcium carbonate, calcium citrate, calcium glubionate, calcium lactate, calcium gluconate, calcium acetate, and combinations thereof. The phosphate salt may be selected from orthophosphates such as monosodium phosphate, disodium phosphate, trisodium phosphate, monopotassium phosphate, dipotassium phosphate, tripotassium phosphate; pyrophosphates such as disodium or dipotassium pyrophosphate, trisodium or tripotassium pyrophosphate, tetrasodium or tetrapotassium pyrophosphate; polyphosphates such as sodium or potassium tripolyphosphate, sodium or potassium tetrapolyphosphate, sodium or potassium hexametaphosphate. The citrate salt may be selected from calcium citrate, potassium citrate, sodium citrate, trisodium citrate, tripotassium citrate, or mixtures thereof. Preferably, the at least one further salt is selected from calcium, phosphate, citrate, sodium, chloride, potassium, metaphosphate, pyrophosphate, tripolyphosphate, longer polyphosphates, and mixtures thereof.

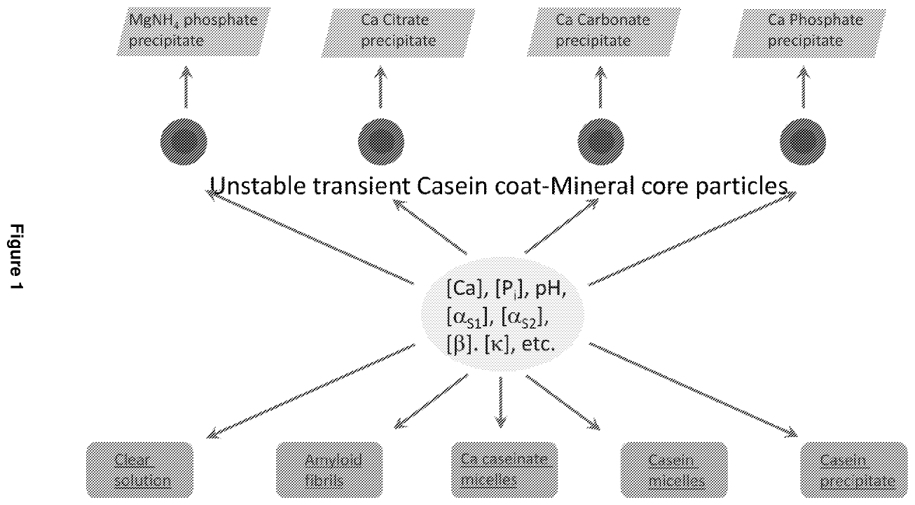

Micelle formation may require solubilization of casein proteins in a solvent such as water. Salts may be added after the solubilization of casein proteins in a solvent. Alternatively, salts and casein proteins may be added to the solution simultaneously. Salts may be added more than once during micelle formation. For instance, calcium salts, phosphate salts and citrate salts may be added at regular intervals or in a continuous titration process and mixed in a solution comprising casein proteins until a micelle formation is achieve. Different salts may be added at different times during the micelle formation process. For instance, calcium salts may be added before the addition of phosphate and citrate salts, or citrate salts may be added before the addition of calcium and phosphate salts, or phosphate salts might be added before the addition of calcium and citrate salts. A preferred method is to add salts incrementally to the protein solution with mixing and pH adjustments to ensure that casein micelle particles are formed in preference to the alternative outcomes outlined in FIG. 1.

Additional components may be added to the casein micelle composition such as fats and sugars. In some embodiments, fat is added to the casein micelle composition. In some embodiments, fats may be essentially free of animal-derived fats. Fats used herein may include plant-based fats such as canola oil, sunflower oil, coconut oil or combinations thereof. The concentration of fats may be about 0% to about 5% in the casein micelle composition. The concentration of fats may be at least 0.5% or about 1%. The concentration of fats may be at most 5%. The concentration of fats may be about 0%, 0.1%, 0.5%, 1%, 2%, 3%, 4% or 5%. The concentration of fats may be from 0 to 0.5%, 0.5% to 1%, 1% to 3%, 1% to 4%, or 1% to 5%. The concentration of fats may be at most 2%, 3%, 4%, or 5%. Sugars used herein may include plant-based disaccharides and/or oligosaccharides. Examples of sugars include sucrose, glucose, fructose, galactose, lactose, maltose, mannose, allulose, tagatose, xylose, and arabinose.

In some embodiments of the method, the calcium sensitive casein protein is recombinantly produced, preferably from a host cell that is selected from the group consisting of bacteria, yeast, and fungi.

The calcium sensitive casein proteins of the present invention may be produced in the same host cell. Alternatively, the calcium sensitive casein proteins may be produced in different host cells.

The casein micelle compositions of the present invention do not comprise kappa casein. The proteins of the present invention therefore do not comprise a protein sequence for kappa casein (SEQ ID NOs: 50-66).

In preferred embodiments, the recombinantly produced casein protein has an amino acid sequence comprising SEQ ID NO. 1-49 (as shown in Table 1) or a variant thereof with at least 80% sequence homology. The protein may have at least 80%, 85%, 90%, 95%, 96%, 97%, 98%, or 99% sequence identity to SEQ ID NO. 1-49 (as shown in Table 1).

The casein micelle compositions of the present invention with additional components may be generated by mixing different components at a temperature from 15° C. to 45° C. For instance, a casein micelle composition with one or more recombinant proteins may be mixed with fats and/or sugars at a temperature of about 15° C., 17° C., 20° C., 22° C., 25° C., 27° C., 30° C., 32° C., 35° C., 37° C., 40° C., 42° C. or 45° C.

### Food Products

The present invention also relates to a food product comprising the casein micelle composition disclosed herein.

In some embodiments, the food product is dairy composition. Preferably, the food product is a cheese composition, a yogurt composition or a milk composition.

A milk composition in accordance with the present invention may comprise other components such as sugars, fats, stabilizers and flavouring agents. The texture of a milk composition as described herein may be comparable to the texture of an animal-derived milk. The taste of a milk composition as described herein may be comparable to an animal-derived milk.

A cheese composition in accordance with the present invention may not comprise any animal-derived components. Cheese compositions as described herein may not comprise any animal-derived dairy-based components, such as animal-derived dairy proteins. The texture of a cheese composition as described herein may be comparable to the texture of a similar type of cheese made using animal-derived dairy derived proteins, such as cheese made from animal milk. The taste of a cheese composition as described herein may be comparable to a similar type of cheese made using animal-derived dairy proteins.

A yogurt composition in accordance with the present invention may comprise other components such as sugars, fats, stabilizers and flavouring agents. The texture of a yogurt composition as described herein may be comparable to the texture of an animal-derived yogurt. The taste of a yogurt composition as described herein may be comparable to an animal-derived yogurt.

### EXAMPLES

The invention will now be further explained and illustrated by reference to the following non-limiting examples.

### Example 1: Expression of Casein Proteins in E. coli

### Constructs Design, Cloning and Transformation

Bovine kappa-casein (variant B) and beta casein (variant A2) were first codon optimised for expression in E. coli, synthesised and cloned into pET-26b (+) using the NdeI/XhoI restriction sites by GenScript®. These plasmids were then transformed into chemically competent E. coli T7 Express cells and grown overnight at 37° C. on LB-agar supplemented with kanamycin for selection.

### Protein Expression and Analysis

Individual colonies were grown at 37° C. in liquid LB supplemented with kanamycin and grown to an OD600=0.6. Target protein expression then proceeded by the addition of 1 mM IPTG for 2.5 hrs. Samples were prepared by pelleting 1 mL of culture, lysing the pellet in 200 μL NEB Express Lysis Reagent, then adding an equal volume of Laemmli buffer (+DTT) to the samples. The sample were analysed using the BioRad stain free SDS-PAGE system, as well as the protein identity confirmed by MS/MS.

### Example 2: Expression of Casein Proteins in P. Pastoris

### Constructs Design, Cloning and Transformation

The genes corresponding to kappa-casein (variant B), beta casein (variant A2), alpha-S1-casein (variant B) and alpha-S2-casein (variant A) were codon-optimised and synthesised by ATUM (California, US). The genes were amplified by PCR with primers from IDT and assembled into the P. pastoris vector pD912-AA (ATUM) using the NEBuilder® HiFi DNA Assembly mix and transformed into DH5a competent cells. Plasmid minipreps were performed and the vectors were verified by Sanger sequencing. Subsequently chemically competent P. pastoris (PPS-9011 ATUM (California, US)) were transformed and grown at 30° C. on YPD-agar containing Zeocin for selection.

### Protein Expression and Analysis

Stabilization phase 1:96 clones were randomly chosen and grown in 300 μL YPD in a 96-well plate at 28 C, 600 rpm, 70% humidity for 24 h.

Stabilization phase 2:3 μL of the previously grown strains were inoculated on YPD+Zeocin (250 ug/mL) and grown with the same culturing conditions for 18 h.

Autoinduction phase: 3 μL of the strains in YPD+Zeo were inoculated in BMGM media and grown with the same culturing conditions for 72 h.

The 96-well plate was then centrifuged at 5000×g for 30 min at 4 C. And carefully 10 μL of supernatant were removed to be analysed using the BioRad stain free SDS-PAGE system and the protein identity confirmed by MS/MS.

### Example 3: Expression of Casein Proteins in Y. Lipolytica

### Constructs Design, Cloning and Transformation

The open reading frames (ORFs) corresponding to kappa-casein (variant B), beta-casein (variant A2) and bovine lactoferrin were codon-optimised using the Benchling (https://www.benchling.com/) codon-optimisation tool. The Y. lipolytica LIP2 secretion signal sequence was added to the 5′-end of each sequence. The codon optimised sequences were synthesised and individually cloned into pUC-GW-amp cloning vectors by Azenta (Massachusetts, US). The ORFs were liberated from each plasmid using Pacl and Swal restriction enzymes, and cloned into corresponding sites of the pHYL1001 vector, after fragment separation and purification. The pHYL1001 vector (synthesised by Azenta) was designed to allow expression of cloned ORFs using the Y. lipolytica TEF1 promoter, uracil auxotrophic selection and zeta-site integration. Plasmids were enumerated in E. coli DH5a cells. Plasmid minipreps were performed and the vectors were verified by Sanger sequencing. Subsequently, plasmids were linearised by NotI restriction digest and transformed into chemically competent Y. lipolytica PO1f (ATCC MYA-2613) cells. Transformants we selected using SD^{-uracil} selective agar plates.

### Protein Expression and Analysis

Purification phase: seven clones were randomly chosen from each plate and streaked onto SD^{-uracil} agar plates and incubated at 28° C. for 48 h.

Preculture phase: a single colony from each clone were inoculated into 50 mL falcon tubes containing 3 mL buffered 2× SD-uracil (with 2% glucose). These were grown for 24 h at 28° C. and shaking at 150 rpm.

Production phase: precultures were used to inoculate 250 mL flasks containing 10 mL of buffered 2× SD^{-uracil} (with 4% glucose) to an OD₆₀₀ of 0.2. Cultures were grown for 72 h at 28° C. and shaking at 300 rpm with continuous oxygen transfer rate monitoring using a TOMs fermentation system (Kuhner AG, Switzerland).

Optical densities were determined for each sample and supernatants obtained by centrifugation at 8000×g for 5 min at 4 C. 10 μL of supernatant were removed to be analysed for secreted proteins using the BioRad stain free SDS-PAGE system and the protein identity confirmed by MS/MS.

### Example 4: Expression of Casein Proteins in B. Subtilis

### Constructs Design, Cloning and Transformation

The genes corresponding to kappa-casein (variant B), beta casein (variant A2), alpha-S1-casein (variant B) and alpha-S2-casein (variant A) were codon-optimised using GenScript and synthesised by GenScript. The genes were amplified by PCR with primers from IDT and assembled into the B. subtilis/E. coli shuttle vector pTTB2 (MoBiTech, Germany) using the NEBuilder® HiFi DNA Assembly mix and transformed into DH5a competent cells. Plasmid minipreps were performed and the vectors were verified by Sanger sequencing. The pTTB2 vector was linearised through EcoRI restriction digestion to remove all sequences required for E. coli propagation. Subsequently chemically competent B. subtilis strain WEA (MoBiTech, Germany) were transformed and grown at 37° C. on 2×YT-agar containing 1% xylose for selection.

### Protein Expression and Analysis

Preculture phase: 96 colonies were inoculated into a deep-well plates containing 300 μL LB media and grown at 37° C. for 18 h and 600 rpm.

Autoinduction phase: 3 μL of the precultured strains were inoculated LS-medium media and grown with the same culturing conditions for 48 h.

The 96-well plate was then centrifuged at 5000×g for 30 min at 4 C. 10 μL of supernatant were removed to be analysed using the BioRad stain free SDS-PAGE system and the protein identity confirmed by MS/MS.

### Example 5: Purification of Bovine Casein Proteins

### Purity Check

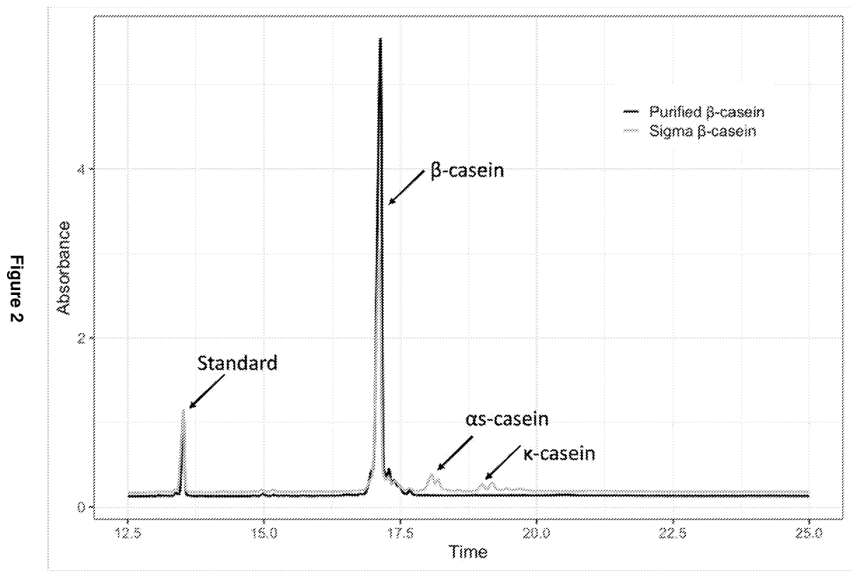

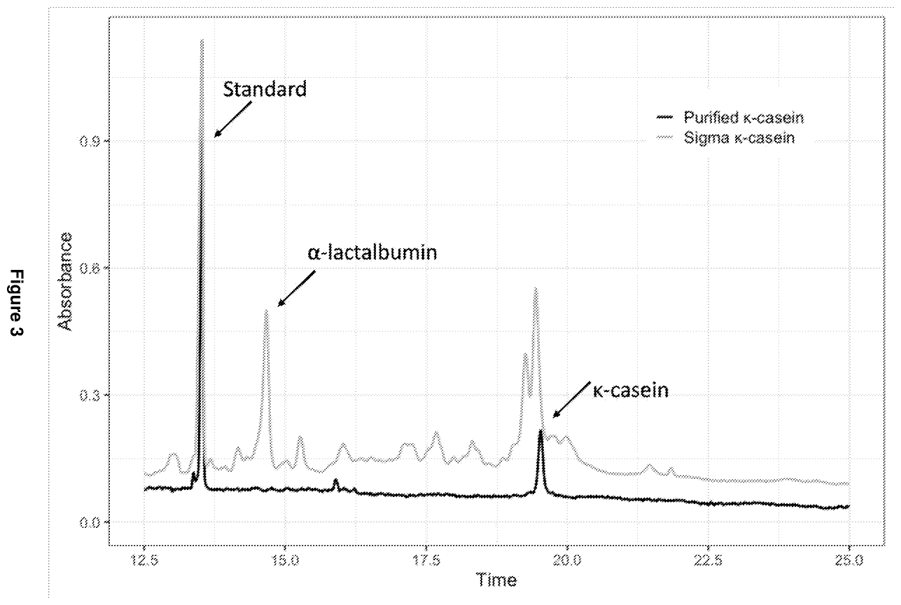

Bovine beta-casein (>98% PAGE purity), kappa-casein (>70% PAGE purity) and alpha-casein (>70% PAGE purity) proteins were purchased from Sigma Aldrich. The proteins were initially analysed by CE-SDS using a Sciex Biophase 8800 to check purity and contamination, in particular to check for kappa-casein contamination in the beta-casein and alpha-casein proteins. The CE-SDS showed that beta-casein contained 87% beta-casein, 9% alpha-casein and 3% kappa-casein (FIG. 2), the kappa-casein contained 52% kappa-casein, 29% alpha lactalbumin, 5% beta lactoglobulin, 5% beta-casein and 5% alpha-casein and 4% other (FIG. 3). Therefore, the proteins were subsequently purified using anion exchange chromatography.

### Anion Exchange Chromatography

Buffer A: 3.3M urea, 20 mM Tris, pH 8.0
Buffer B: 3.3M urea, 20 mM Tris, 1M NaCl, pH 8.0

500 mg Sigma protein or sodium caseinate produced from raw milk was dissolved in 50 mL buffer A containing 20 mM DTT and subsequently filtered through a 0.2 μm syringe filter. A HiPrep Q HP 16/10 was connected to a Äkta Pure 150 and equilibrated with 5 CV of buffer A before loading of the protein. The column was then washed with 4 CV of buffer A before a linear gradient to 40% buffer B over 10CV was applied with fractions collected. This provided very good separation of all the casein proteins which were reanalysed by CE-SDS and showed >99% purity.

### Example 6: 38KX Casein Micelle Manufacture and Analysis

Casein micelles were made using re-purified bovine beta-casein and kappa-casein from Sigma Aldrich, keeping the beta-casein concentration constant at 8 g/L⁻¹ and varying the kappa-casein concentration from 1-10 g/L with the compositions shown in Table 2.

TABLE 2
Casein micelle compositions

| Beta-casein | Kappa-casein | Pi | Ca | NaCl | NaN₃ |
|---|---|---|---|---|---|
| (g/L⁻¹) | (g/L⁻¹) | (mM) | (mM) | (mM) | (mM) |
| 8 | 2 | 7.1 | 6.5 | 36 | 1.5 |
| 8 | 3 | 7.1 | 6.5 | 36 | 1.5 |
| 8 | 5 | 7.1 | 6.5 | 36 | 1.5 |
| 8 | 10 | 7.1 | 6.5 | 36 | 1.5 |

The casein micelles were made by first dissolving the protein in 18.2MΩ-cm ultrapure water and then subsequently titrating the minerals into the protein solution. Sodium azide was added to stop bacterial growth for subsequent casein micelle stability studies.

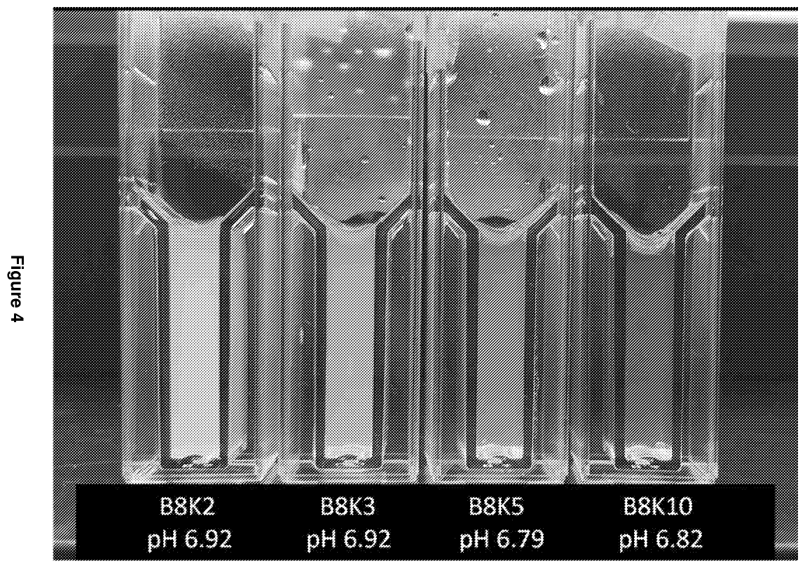

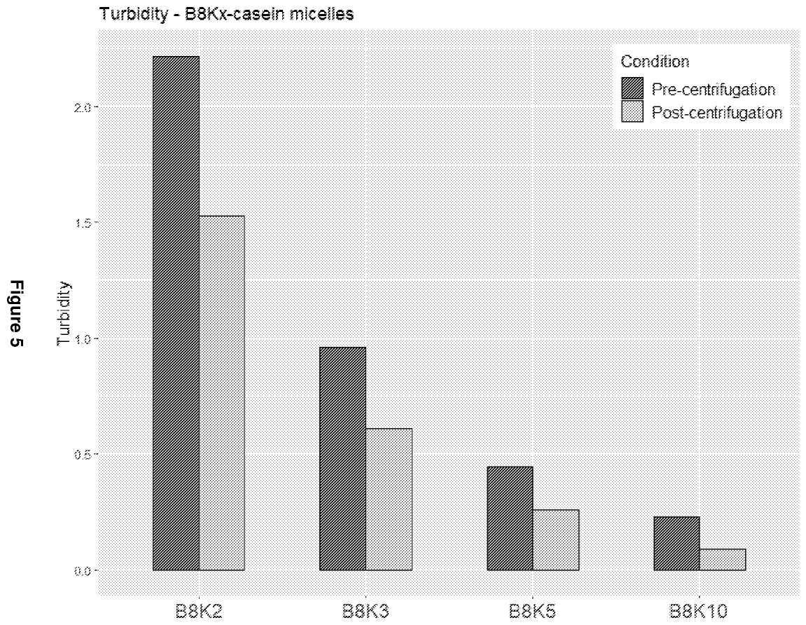

Following micelle formation, solutions were left for a further 12 hrs to ensure stable micelles had formed. The micelle solutions are shown in FIG. 4 and as can seen, with increasing kappa-casein concentration, the whiteness of the solution decreases as is expected with properly formed casein micelles as kappa-casein concentration is related to casein micelle size and hence the scattering of light. Turbidity of the solutions was also measured pre- and post-centrifugation at 1000×g for 3 mins (FIG. 5). If casein micelles have formed correctly then the turbidity measurements should be within ˜50% for the pre- and post-centrifugation readings. Turbidity was measured at 600 nm using a BMG Labtech CLARIOstar Plus microplate reader by diluting the samples 4× in the ultra-filtrate of each sample. Ultrafiltrates were produced by centrifuging 6 mL of casein micelle sample in a Sartorius Vivaspin 6 5,000 MWCO PES centrifuge filter. This same filtrate was used to dilute the samples for NanoSight particle tracking analysis (NTA).

Next the casein micelle size of the solutions were measured using a Malvern NanoSight NS300 equipped with a NanoSight Sample Assistant by first diluting the samples either by 1000× (B8K2) or 10000× (B8K3/5/10) using the individual ultra-filtrate of each sample.

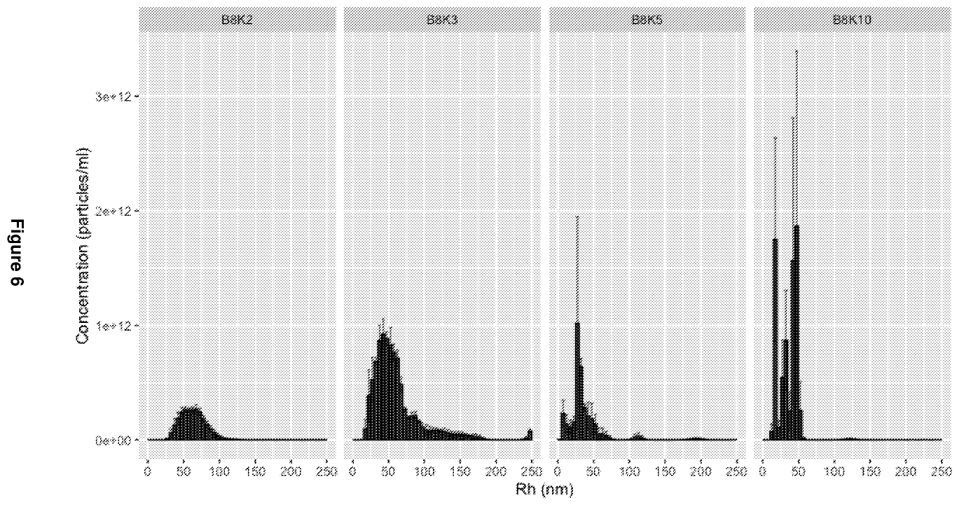

As can be seen, there is an inverse relationship between the amount of kappa-casein, turbidity (FIG. 5) and casein micelle size (FIG. 6).

Mineral analysis using of the ultrafiltrates produced for the NTA analysis were measured using a Perkin Elmer Nexion 259X Inductively Coupled Plasma Mass Spectrometer (ICPMS). Table 3 contains the concentration of Pi and Ca measured in the ultrafiltrates of the different micelle compositions, showing that micelles had correctly formed and bound the remainder of the Pi and Ca.

TABLE 3
Ultrafiltrate (UF) mineral analysis by ICPMS

| Beta- | Kappa- | | | | |
|---|---|---|---|---|---|
| casein | casein | Pi (mM) | Ca (mM) | Pi (mM) | Ca (mM) |
| (g/L⁻¹) | (g/L⁻¹) | total | total | UF | UF |
| 8 | 2 | 7.1 | 6.5 | 4.13 | 2.76 |
| 8 | 3 | 7.1 | 6.5 | 5.33 | 3.41 |
| 8 | 5 | 7.1 | 6.5 | 5.52 | 4.10 |
| 8 | 10 | 7.1 | 6.5 | 5.30 | 2.92 |

### Example 7: Stable Artificial Casein Micelles Made of Bovine Beta Casein without Kappa Casein

Beta-casein micelle compositions were made where the total protein concentration was kept at 10 g L⁻¹, pH was kept constant at 7.00, whilst the concentration of calcium, phosphate and NaCl was varied according to Table 4.

TABLE 4
Beta-casein only micelle compositions

| Sample | Beta-casein | Pi | Ca | NaCl | NaN₃ | |
|---|---|---|---|---|---|---|
| # | (g L⁻¹) | (mM) | (mM) | (mM) | (mM) | pH |
| 1 | 10 | 25.4 | 4 | 25.2 | 1.5 | 7.00 |
| 2 | 10 | 25.6 | 4.6 | 23.9 | 1.5 | 7.00 |
| 3 | 10 | 25.9 | 5.2 | 22.4 | 1.5 | 7.00 |

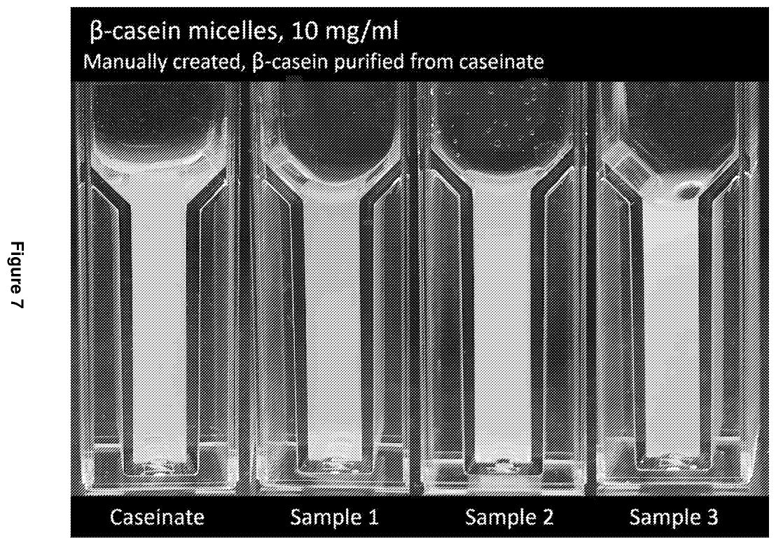

Samples were made by first dissolving the beta-casein in 18.2MΩ-cm ultrapure water followed by titration of the minerals so as to achieve the desired final concentrations. The samples were allowed to equilibrate with mixing for 2 hrs before analysis (FIG. 7).

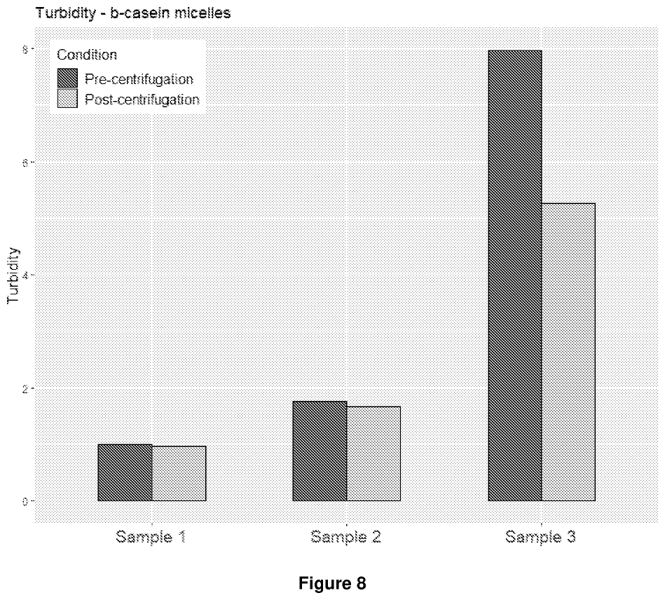

The first analysis to test a stable casein micelle had been formed was the measurement pre- and post-centrifugation. As can been seen in FIG. 8, stable casein micelles where formed as the pre- and post-centrifugation turbidity was <50%. Turbidity increases with increasing Ca/Pi concentration.

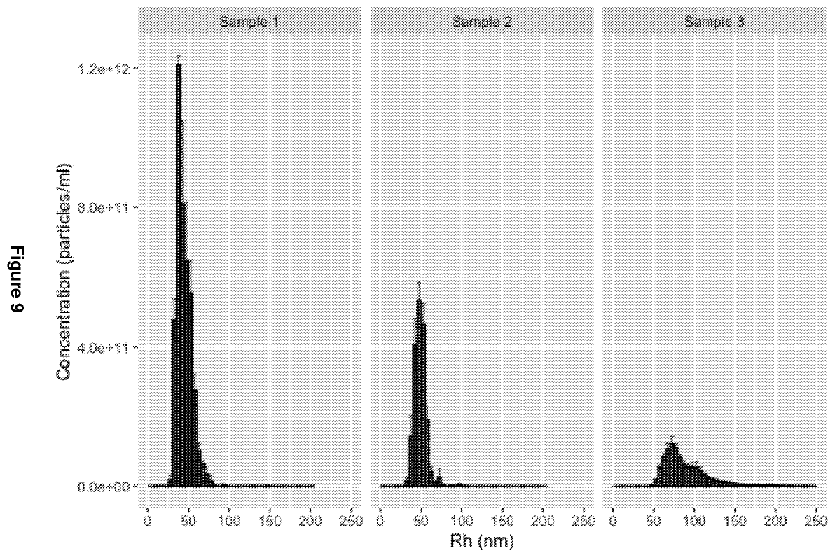

Next nanoparticle tracking analysis was used to measure casein micelle size and showed micelle size increases with a decrease in the number of particles with an increase in the Ca/Pi concentration (FIG. 9).

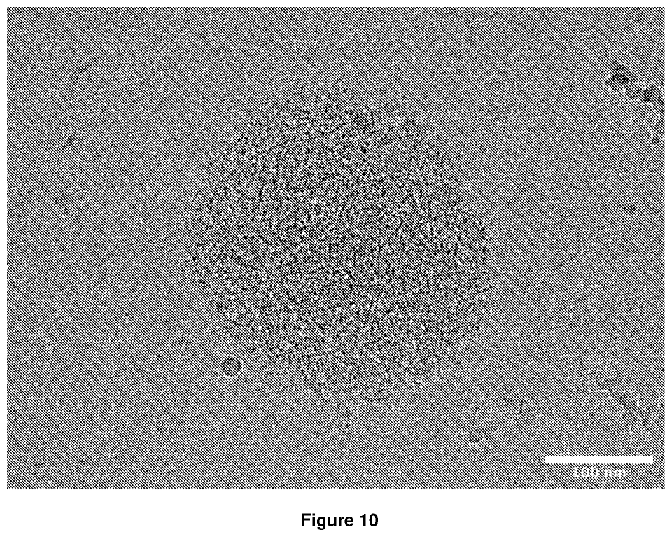

Cryo-transmission electron microscopy (cryo-TEM) was used to visualise the formed casein micelles. FIG. 10 shows Sample 2 casein micelles which show properly formed casein micelles with calcium-phosphate nanoclusters clearly visible. These single-casein protein micelles have a remarkably similar architecture to native cows' milk casein micelles from literature such as Day, L., J. K. Raynes, A. Leis, L. H. Liu, and R. P. W. Williams. 2017. Food Hydrocolloids 69:150-163.

Mineral analysis using of the ultrafiltrates produced for the NTA analysis were measured using a Perkin Elmer Nexion 259X Inductively Coupled Plasma Mass Spectrometer (ICPMS). Table 5 contains the concentration of Pi and Ca measured in the ultrafiltrates of the different micelle compositions, showing that micelles had correctly formed and bound the remainder of the Pi and Ca.

TABLE 5
Beta-casein only ultrafiltrate (UF) mineral analysis by ICPMS

| Sample | Beta-casein | Pi (mM) | Ca (mM) | Pi (mM) | Ca (mM) |
|---|---|---|---|---|---|
| # | (g L⁻¹) | Total | Total | UF | UF |
| 1 | 10 | 25.4 | 4 | 24.32 | 0.300 |
| 2 | 10 | 25.6 | 4.6 | 19.55 | 0.561 |
| 3 | 10 | 25.9 | 5.2 | 19.64 | 0.300 |

### Example 8: Stable Artificial Casein Micelles Made of Bovine Alpha-s-Casein without Kappa Casein

Bovine alpha-s-casein, containing both alpha-s1-casein and alpha-s2-casein was purified from sodium caseinate using anion exchange chromatography as previously described. Capillary electrophoresis was used to confirm purity and ensure the absence of kappa-casein.

Alpha-s-casein micelle compositions were made where the total protein concentration was kept at 10 g L⁻¹, pH was kept constant at 7.00, whilst the concentration of calcium, phosphate and NaCl was varied according to Table 6.

TABLE 6
alpha-s-casein only micelle compositions

| | alpha-s- | | | | | |
|---|---|---|---|---|---|---|
| Sample | casein | Calcium | Phosphate | NaCl | NaN₃ | |
| # | (g L⁻¹) | (mM) | (mM) | (mM) | (mM) | pH |
| 1 | 10 | 4.3 | 25.7 | 22.08 | 1.5 | 7.00 |
| 2 | 10 | 6.75 | 27.10 | 16.55 | 1.5 | 7.00 |

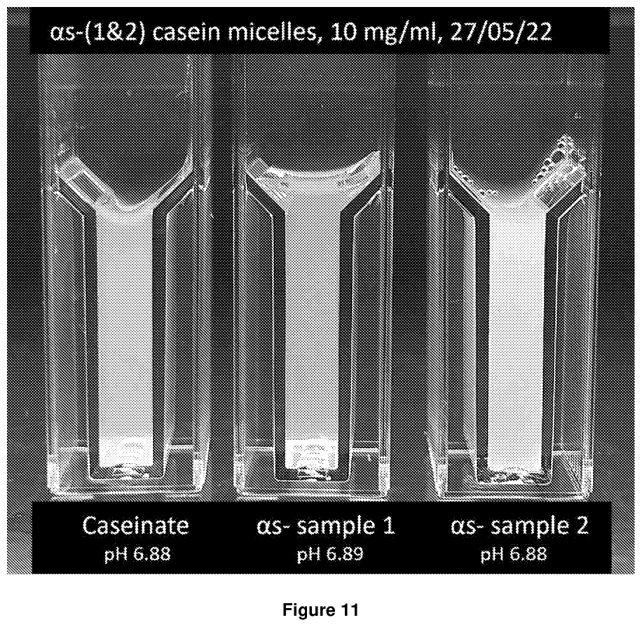

Samples were made by first dissolving the alpha-s-casein in 18.2MΩ-cm ultrapure water followed by titration of the minerals so as to achieve the desired final concentrations. The samples were allowed to equilibrate with mixing for 2 hrs before analysis (FIG. 11) and sodium caseinate micelles were made in parallel as a control.

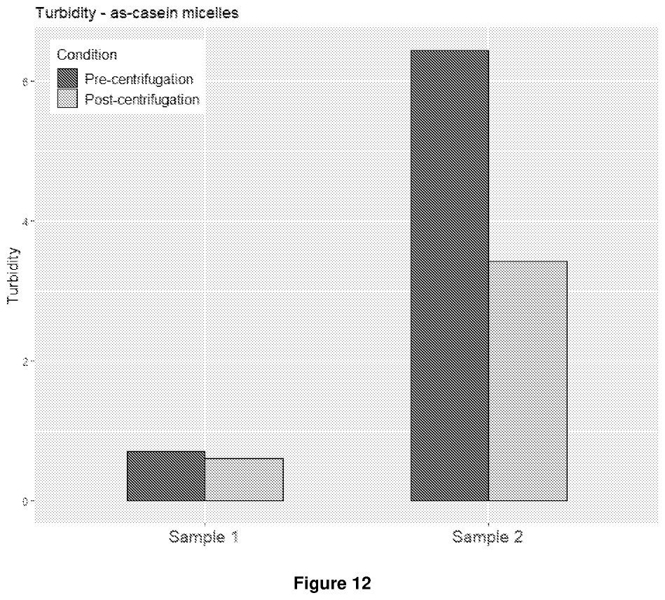

The first analysis to test a stable casein micelle had been formed was the measurement pre- and post-centrifugation. As can been seen in FIG. 12, stable casein micelles were formed as the pre- and post-centrifugation turbidity was <50%. Turbidity increases with increasing Ca/Pi concentration.

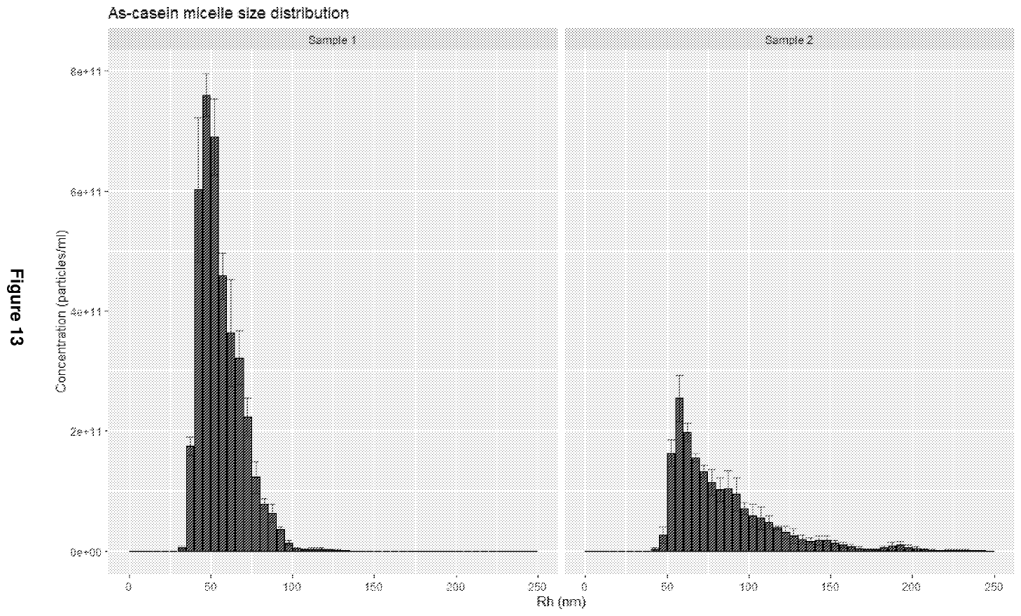

Next nanoparticle tracking analysis was used to measure casein micelle size (FIG. 13) which showed an increase in casein micelle size with an increased Ca and Pi concentration.

### Prophetic Example 1: Milk Beverage Compositions Containing Single Protein Casein Micelles

Example compositions for milk beverages containing single-casein micelles are shown in Table 7. It should be noted that whilst only a single composition per protein is expressed here, many different compositions for a milk beverage are possible based on this patent application and a person skilled in the art will recognise this. Single casein micelles are first formed by dissolving the casein protein powder into the required volume of water and then titrating the minerals into the solution. Once micelles have formed, beta-lactoglobulin and maltose is added to the solution, followed by pre-warmed fat components. The solution is then homogenized, pasteurized and bottled prior to consumption.

TABLE 7
Compositions for milk beverages

| Beta- | Beta-lacto- | | | | | | |
|---|---|---|---|---|---|---|---|
| casein | globulin | Calcium | Phosphate | NaCl | | Maltose | Fat |
| (g L⁻¹) | (g L⁻¹) | (mM) | (mM) | (mM) | pH | (g L⁻¹) | (g L⁻¹)* |
| 26.4 | 6.6 | 11.5 | 22.69 | 20.81 | 7.00 | 30 | 35 |

| Alpha-s1- | Beta-lacto- | | | | | | |
|---|---|---|---|---|---|---|---|
| casein | globulin | Calcium | Phosphate | NaCl | | Maltose | Fat |
| (g L⁻¹) | (g L⁻¹) | (mM) | (mM) | (mM) | pH | (g L⁻¹) | (g L⁻¹)* |
| 26.4 | 6.6 | 19.5 | 32.5 | 3 | 7.00 | 30 | 35 |
| Alpha-s2- | Beta-lacto- | | | | | | |
| casein | globulin | Calcium | Phosphate | NaCl | | Maltose | Fat |
| (g L⁻¹) | (g L⁻¹) | (mM) | (mM) | (mM) | pH | (g L⁻¹) | (g L⁻¹)* |
| 26.4 | 6.6 | 19.5 | 32.5 | 3 | 7.00 | 30 | 35 |

*Fats can be a mixture of any plant-based fats including but not limited to coconut oil, canola oil, sunflower oil, mono and di glycerides, tributyrin, butyric and hexanoic acid, phospholipids

### Prophetic Example 2: Yoghurt Compositions Containing Single Protein Casein Micelles

Example compositions for yoghurts containing single-casein micelles are shown in Table 8. It should be noted that whilst only a single composition per protein is expressed here, many different compositions for a yoghurt are possible based on this patent application and a person skilled in the art will recognise this. Single casein micelles are first formed by dissolving the casein protein powder into the required volume of water and then titrating the minerals into the solution. Once micelles have formed, beta-lactoglobulin and maltose is added to the solution, followed by pre-warmed fat components. Next the milk base is heated to 85° C. to denature the whey proteins followed by cooling to 40° C. before addition of a lactic acid starter culture (e.g. Lactobacillus strain). Fermentation is then allowed to proceed for 4-8 hours before cooling to 4° C. and packaging.

TABLE 8
Compositions for yoghurts

| Beta- | Beta-lacto- | | | | | |
|---|---|---|---|---|---|---|
| casein | globulin | Calcium | Phosphate | NaCl | Maltose | Fat |
| (g L⁻¹) | (g L⁻¹) | (mM) | (mM) | (mM) | (g L⁻¹) | (g L⁻¹)* |
| 26.4 | 6.6 | 11.5 | 22.69 | 20.81 | 30 | 35 |

| Alpha-s1- | Beta-lacto | | | | | |
|---|---|---|---|---|---|---|
| casein | globulin | Calcium | Phosphate | NaCl | Maltose | Fat |
| (g L⁻¹) | (g L⁻¹) | (mM) | (mM) | (mM) | (g L⁻¹) | (g L⁻¹)* |
| 26.4 | 6.6 | 19.5 | 32.5 | 3 | 30 | 35 |

| Alpha-s2- | Beta-lacto- | | | | | |
|---|---|---|---|---|---|---|
| casein | globulin | Calcium | Phosphate | NaCl | Maltose | Fat |
| (g L⁻¹) | (g L⁻¹) | (mM) | (mM) | (mM) | (g L⁻¹) | (g L⁻¹)* |
| 26.4 | 6.6 | 19.5 | 32.5 | 3 | 30 | 35 |

*Fats can be a mixture of any plant-based fats including but not limited to coconut oil, canola oil, sunflower oil, mono and di glycerides, tributyrin, butyric and hexanoic acid, phospholipids

### Prophetic Example 3: Fresh Cheese Compositions Containing Single Protein Casein Micelles

Example compositions for cheeses containing single-casein micelles are shown in Table 9. It should be noted that whilst only a single composition per protein is expressed here, many different compositions for a cheese are possible based on this patent application and a person skilled in the art will recognise this. Single casein micelles are first formed by dissolving the casein protein powder into the required volume of water and then titrating the minerals into the solution. Once micelles have formed, maltose is added to the solution, followed by pre-warmed fat components. Next the milk base is warmed to 38° C. and a rennet substitute (as there is no kappa casein) such as microbial pepsin is added based on manufacturer's instructions. The milk is then left for 30 min to coagulate. Once coagulated, cut the curds into roughly 2.5 cm cubes and gently scoop out and place into a cheese mould to remove the moisture.

TABLE 9
Compositions for cheeses

| Beta-casein | Calcium | Phosphate | NaCl | Maltose | Fat |
|---|---|---|---|---|---|
| (g L⁻¹) | (mM) | (mM) | (mM) | (g L⁻¹) | (g L⁻¹)* |
| 26.4 | 11.5 | 22.69 | 20.81 | 30 | 35 |

| Alpha-s1- | Calcium | Phosphate | NaCl | Maltose | Fat |
|---|---|---|---|---|---|
| casein (g L⁻¹) | (mM) | (mM) | (mM) | (g L⁻¹) | (g L⁻¹)* |
| 26.4 | 19.5 | 32.5 | 3 | 30 | 35 |
| Alpha-s2- | Calcium | Phosphate | NaCl | Maltose | Fat |
| casein (g L⁻¹) | (mM) | (mM) | (mM) | (g L⁻¹) | (g L⁻¹)* |
| 26.4 | 19.5 | 32.5 | 3 | 30 | 35 |

*Fats can be a mixture of any plant-based fats including but not limited to coconut oil, canola oil, sunflower oil, mono and di glycerides, tributyrin, butyric and hexanoic acid, phospholipids ***

In some embodiments, an invention is provided according to the following numbered clauses:
1. A casein micelle composition containing amorphous calcium phosphate (CaP), wherein the casein micelle comprises at least one calcium sensitive casein, and wherein the casein micelle does not contain κ-casein.
2. The casein micelle composition according to clause 1, wherein the casein micelle comprises:
   (i) only αₛ₁-casein;
   (ii) only αₛ₂-casein;
   (iii) only β-casein;
   (iv) a mixture of αₛ₁- and β-casein;
   (v) a mixture of αₛ₂- and β-casein;
   (vi) a mixture of αₛ₁- and αₛ₂-casein; or
   (vii) a mixture of αₛ₁-, αₛ₂- and β-casein.
3. The casein micelle composition according to clause 2, wherein the casein micelle composition contains sufficient CaP to bind between about 5% and 100% of the casein.
4. The casein micelle composition according to any one of clauses 1 to 3, wherein the total casein concentration in the composition is between about 0.5-100 g L⁻¹, preferably between around 5 to 50 g L 1, preferably around 30 g L⁻¹.
5. The casein micelle composition according to any one of clauses 1 to 4, wherein the pH of the composition is between about 5.5 to 8.0, preferably about pH 6.7.
6. The casein micelle composition according to any one of clauses 2 to 5, wherein when the casein micelle composition comprises (i) only αₛ₁-casein, the concentration of calcium in the composition is between 1.48 and 150.00 mM, preferably between 2.72 and 38.58 mM.
7. The casein micelle composition according to any one of clauses 2 to 5, wherein when the casein micelle composition comprises (i) only αₛ₁-casein, the concentration of phosphate in the composition is between 23.5 and 58.40 mM, preferably between 24.42 and 43.66 mM.
8. The casein micelle composition according to any one of clauses 2 to 5, wherein when the casein micelle composition comprises (i) only αₛ₂-casein, the concentration of calcium in the composition is between 1.48 and 150.00 mM, preferably between 2.72 and 38.58 mM.
9. The casein micelle composition according to any one of clauses 2 to 5, wherein when the casein micelle composition comprises (i) only αₛ₂-casein, the concentration of phosphate in the composition is between 23.5 and 58.40 mM, preferably between 24.42 and 43.66 mM.
10. The casein micelle composition according to any one of clauses 2 to 5, wherein when the casein micelle composition comprises (i) only β-casein, the concentration of calcium in the composition is between 1.3 and 34.0 mM, preferably between 1.81 and 7.69 mM.
11. The casein micelle composition according to any one of clauses 2 to 5, wherein when the casein micelle composition comprises (i) only β-casein, the concentration of phosphate in the composition is between 24.54 and 41.0 mM, preferably between 24.54 and 36.1 mM.
12. The casein micelle composition according to any one of clauses 2 to 5, wherein the composition comprises a mixture of calcium sensitive caseins; and the calcium and phosphate concentrations are a combination of the ranges for the individual casein components but combined in proportion to the mole fraction of each casein in the mixture.
13. The casein micelle composition according to any one of clauses 1 to 12, wherein the calcium sensitive casein protein is recombinantly produced.
14. The casein micelle composition according to clause 13, wherein the recombinantly produced casein protein(s) are produced from a host cell that is selected from the group consisting of bacteria, yeast, and fungi.
15. The casein micelle composition according to any one of clauses 1 to 14, wherein the casein micelle composition remains dispersed under centrifugation at 4000×g for 3 mins.
16. The casein micelle composition according to any one of clauses 1 to 14, wherein the casein micelle composition remains dispersed when pasteurised for 15 s at 72° C.
17. The casein micelle composition according to any one of clauses 1 to 14, wherein the casein micelle composition remains dispersed when pasteurised for 1-4 seconds at 135° C.
18. The casein micelle composition according to any one of clauses 13 to 17, wherein the recombinantly produced casein protein has an amino acid sequence comprising SEQ ID NO. 1-49 or a variant thereof with at least 80% sequence homology.
19. The casein micelle composition according to any one of clauses 14 to 18, wherein the bacterial host cell is selected from the group consisting of Lactococci sp., Lactococcus lactis, Bacillus subtilis, Bacillus amyloliquefaciens, Bacillus licheniformis and Bacillus megaterium, Brevibacillus chohinensis, Mycobacterium smegmatic, Rhodococcus erythroplois and Corynebacterium glutamicum, Lactobacilli sp., Lactobacillus fermentum, Lactobacillus casei, Lactobacillus acidophilus, Lactobacillus plantarum, Synechocystis sp. 6803 and E. coli.
20. The casein micelle composition according to any one of clauses 14 to 18, wherein the yeast host cell is selected from the group consisting of Kluyveromyces sp., Pichia sp., Saccharomyces sp., Tetrahynena sp., Yarrowia sp., Hansenula sp., Blastobotrys sp., Candida sp., Zygosaccharomyces sp., or Debaryomyces sp.
21. The casein micelle composition according to any one of clauses 14 to 18, wherein the fungal host cell is selected from the group consisting of any Aspergillus sp. (such as Aspergillus nidulans, Aspergillus niger, Aspergillus oryzae), Trichoderma reesei, Chrysosporium lucknowense, Fusarium sp., Fusarium granineum, Fusarium venenatum, Physcoinitrella patens, and Neurospora crassa.
22. The casein micelle composition according to any one of clauses 1 to 21, wherein the casein proteins comprise an amino acid sequence of any one of the group consisting of cow, goat, sheep, buffalo, camel, horse, bison, human, donkey, chimpanzee, rabbit, mouse, guinea pig, brush-trailed possum, duckbill platypus, Australian echidna, wallaby, zebu, or mixtures thereof.
23. The casein micelle composition according to any one of clauses 1 to 22, wherein the casein micelle further comprises one or more non-casein proteins.
24. The casein micelle composition according to clause 23, wherein the non-casein protein is selected from the group consisting of osteopontin, dentin matrix protein, matrix extracellular phosphoglycoprotein, bone sialoprotein, dentin sialophosphoprotein, amelogenin, statherin, starmaker or otolith matrix macromolecule-64 and related homologs, calcium-binding proteins, secretory calcium-binding phosphoproteins (SCPPs) and mixtures thereof.
25. A method of producing a casein micelle composition according to any one of clauses 1 to 24, comprising combining at least one calcium sensitive casein protein with at least one salt under conditions wherein the at least one calcium sensitive casein protein forms a casein micelle in a liquid colloid, wherein the casein micelle does not include κ-casein protein.
26. The method according to clause 25, wherein the at least one salt is a calcium salt and/or a phosphate salt.
27. The method according to clause 26, further comprising the addition of at least one further salt selected from calcium salt, phosphate salt, sodium chloride, potassium chloride, citrate, metaphosphate, pyrophosphate, tripolyphosphate, longer polyphosphates, and mixtures thereof.
28. A food product comprising the casein micelle composition according to any one of clauses 1 to 24.
29. The food product according to clause 28, wherein the food product is dairy composition, preferably a cheese composition, a yogurt composition or a milk composition.

Those skilled in the art will appreciate that the invention described herein is susceptible to variations and modifications other than those specifically described. It is understood that the invention includes all such variations and modifications which fall within the spirit and scope of the present invention.

1. A casein micelle composition comprising casein micelle particles containing amorphous calcium phosphate (CaP), wherein the casein micelle particles comprise at least one calcium sensitive casein, and wherein the casein micelle particles do not contain κ-casein.

2. The casein micelle composition according to claim 1, wherein the casein micelle particles comprise:
(i) only αₛ₁-casein;
(ii) only αₛ₂-casein;
(iii) only β-casein;
(iv) a mixture of αₛ₁- and β-casein;
(v) a mixture of αₛ₂- and β-casein;
(vi) a mixture of αₛ₁- and αₛ₂-casein; or
(vii) a mixture of αₛ₁-, αₛ₂- and β-casein.

3. The casein micelle composition according to claim 2, wherein the casein micelle composition contains sufficient CaP to bind between about 5% and 100% of the casein.

4. The casein micelle composition according to claim 1, wherein the total casein concentration in the composition is about 0.5-100 g/L.

5. The casein micelle composition according to claim 1, wherein the pH of the composition is about 5.5 to 8.0.

6. The casein micelle composition according to claim 2, wherein when the casein micelle particles comprise (i) only αₛ₁-casein, the concentration of calcium in the composition is between 1.48 to 150.00 mM, preferably between 2.72 and 38.58 mM.

7. The casein micelle composition according to claim 2, wherein when the casein micelle particles comprise (i) only αₛ₁-casein, the concentration of phosphate in the composition is 23.5 to 58.40 mM.

8. The casein micelle composition according to claim 2, wherein when the casein micelle particles comprise (ii) only αₛ₂-casein, the concentration of calcium in the composition is 1.48 to 150.00 mM.

9. The casein micelle composition according to claim 2, wherein when the casein micelle particles comprise (ii) only αₛ₂-casein, the concentration of phosphate in the composition is 23.5 to 58.40 mM.

10. The casein micelle composition according to claim 2, wherein when the casein micelle particles comprise (iii) only β-casein, the concentration of calcium in the composition is 1.3 to 34.0 mM.

11. The casein micelle composition according to claim 2, wherein when the casein micelle particles comprise (iii) only β-casein, the concentration of phosphate in the composition is 24.54 to 41.0 mM.

12. The casein micelle composition according to claim 2, wherein the casein micelle particles comprise a mixture of calcium sensitive caseins; and the calcium and phosphate concentrations are a combination of the ranges for the individual casein micelle particle components but combined in proportion to the mole fraction of each casein in the mixture.

13. The casein micelle composition according to claim 1, wherein the calcium sensitive casein protein has been recombinantly produced in a host cell selected from bacteria, yeast, and fungi host cells.

14. (canceled)

15. The casein micelle composition according to claim 1, wherein the casein micelle composition remains dispersed when subjected to conditions selected from centrifugation at 4000×g for 3 mins, pasteurization for 15 s at 72° C., and pasteurization for 1-4 seconds at 135° C.

16. (canceled)

17. The casein micelle composition according to claim 13, wherein the recombinantly produced casein protein has an amino acid sequence comprising the amino acid sequence of any one of SEQ ID NO. 1-49 or a variant thereof with at least 80% sequence homology thereto.

18. The casein micelle composition according to claim 13, wherein when:
(a) the host cell is a bacterial host cell, the bacterial host cell is selected from the group consisting of Lactococci sp., Lactococcus lactis, Bacillus subtilis, Bacillus amyloliquefaciens, Bacillus licheniformis and Bacillus megaterium, Brevibacillus chohinensis, Mycobacterium smegmatic, Rhodococcus erythroplois and Corynebacterium glutamicum, Lactobacilli sp., Lactobacillus fermentum, Lactobacillus casei, Lactobacillus acidophilus, Lactobacillus plantarum, Synechocystis sp. 6803, and E. coli;
(b) the host cell is a yeast host cell, the yeast host cell is selected from the group consisting of Kluyveromyces sp., Pichia sp., Saccharomyces sp., Tetrahynena sp., Yarrowia sp., Hansenula sp., Blastobotrys sp., Candida sp., Zygosaccharomyces sp., or Debaryomyces sp; or
(c) the host cell is a fungal host cell, the fungal host cell is selected from the group consisting of any Aspergillus sp. (such as Aspergillus nidulans, Aspergillus niger, Aspergillus oryzae), Trichoderma reesei, Chrysosporium lucknowense, Fusarium sp., Fusarium granineum, Fusarium venenatum, Physcoinitrella patens, and Neurospora crassa.

19. The casein micelle composition according to claim 1, wherein the casein proteins comprise an amino acid sequence of a casein protein from any animal selected from cow, goat, sheep, buffalo, camel, horse, bison, human, donkey, chimpanzee, rabbit, mouse, guinea pig, brush-trailed possum, duckbill platypus, Australian echidna, wallaby, zebu, or mixtures thereof.

20. The casein micelle composition according to claim 1, wherein the casein micelle particle further comprises one or more non-casein proteins selected from the group consisting of osteopontin, dentin matrix protein, matrix extracellular phosphoglycoprotein, bone sialoprotein, dentin sialophosphoprotein, amelogenin, statherin, starmaker, otolith matrix macromolecule-64, and related homologs, calcium-binding proteins, secretory calcium-binding phosphoproteins (SCPPs) and mixtures thereof.

21. A method of producing a casein micelle composition according to claim 1, comprising combining at least one calcium sensitive casein protein with at least one salt under conditions wherein the at least one calcium sensitive casein protein forms a casein micelle particle in a liquid colloid, wherein the casein micelle does not include κ-casein protein, wherein the at least one salt is one or more of a calcium salt and a phosphate salt.

22. (canceled)

23. The method according to claim 21, further comprising the addition of at least one further salt selected from calcium salt, phosphate salt, sodium chloride, potassium chloride, citrate, metaphosphate, pyrophosphate, tripolyphosphate, longer polyphosphates, and mixtures thereof.

24. A dairy food product comprising the casein micelle composition according to claim 1.

25. The dairy food product according to claim 24, wherein the food product is a cheese composition, a yogurt composition or a milk composition.
